(19) United States
(12) Patent Application Publication
(10) Pub. No.: US 2023/0141254 A1
(43) Pub. Date: May 11, 2023

(54) COSMETIC COMPOSITIONS COMPRISING HIGH AMOUNTS OF RETINOL

(71) Applicant: L'OREAL, Paris (FR)

(72) Inventors: Jonathan James FAIG, Sayreville, NJ (US); Yon Jae Yoon, Roselle, NJ (US); Susan Halpern Chirch, Basking Ridge, NJ (US); Angelike Galdi, Westfield, NJ (US)

(21) Appl. No.: 18/050,593

(22) Filed: Oct. 28, 2022

Related U.S. Application Data
(60) Provisional application No. 63/273,966, filed on Oct. 31, 2021.

(30) Foreign Application Priority Data
Jan. 31, 2022 (FR) 2200829

(51) Int. Cl. A61K 8/67; A61K 8/36; A61K 8/92; A61K 8/25; A61K 8/60; A61K 8/34; A61K 8/06; A61K 8/89; A61Q 19/00

(52) U.S. Cl. CPC A61K 8/671; A61K 8/25; A61K 8/062; A61K 8/89; A61K 8/92; A61K 8/342; A61K 8/361; A61K 8/604; A61Q 19/00

(57) ABSTRACT

The present disclosure relates to stable cosmetic compositions containing high amounts of retinol. The cosmetic compositions further include, among other things, a plurality of nonionic emulsifiers, fatty alcohols, fatty compounds, thickening polymers, and water. Methods for solubilizing high amounts of retinol and methods of treating the skin with the cosmetic compositions is also described. Methods for stabilizing the cosmetic compositions containing high amounts of retinol and methods for preserving the high amounts of retinol are also described. The present disclosure also describes methods for treating the with the cosmetic compositions.

### CROSS-REFERENCE TO RELATED APPLICATIONS

This application claims benefit of U.S. Serial No. 63/273,966 filed Oct. 31, 2021, and benefit of French Application No. FR 2200829, filed on Jan. 31, 2022, which are incorporated herein by reference in their entirety.

### FIELD OF THE DISCLOSURE

The instant disclosure relates to stable cosmetic compositions that include high amounts of retinol; and to methods for stabilizing cosmetic compositions containing high amounts of retinol. The instant disclosure also describes methods for treating skin with the cosmetic compositions.

### SUMMARY

In an aspect, the present disclosure is directed to, among other things, cosmetic compositions including a surprisingly high amount of retinol. Retinol provides a myriad of cosmetic benefits to the skin but has historically been very difficult to stabilize, especially in high amounts. Retinol also is susceptible to degradation and therefore is difficult to incorporate into compositions without it degrading over time. The inventors developed, among other things, surprisingly stable compositions that include high amounts of retinol, which is preserved (resists degradation). Due to the high amounts of retinol, the cosmetic compositions provide exceptional cosmetic properties to the skin, for example, boosting collagen and/or elastin production, reducing the appearance of fine lines, wrinkles, and uneven skin tone, tightening the skin, prevent and/or treat acne, decrease the development of melanin, and improve and brighten complexion.

In an aspect, the present disclosure is directed to, among other things, a cosmetic composition in the form of an oil in water emulsion. In an embodiment, the cosmetic composition includes:
(a) retinol;
(b) a plurality of nonionic emulsifiers comprising:
   (i) one or more ethoxylated fatty acids; and
   (ii) one or more nonionic emulsifiers having a Hydrophile-Lipophile Balance (HLB) from about 9 to about 12;
   (iii) optionally, one or more glyceryl esters;
   (iv) optionally, one or more additional nonionic emulsifiers;
(c) one or more fatty alcohols;
(d) one or more fatty compounds;
(e) one or more thickening polymers; and
(f) water.

Nonlimiting examples of ethoxylated fatty acids include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the INCI names: PEG-9 laurate to PEG-50 laurate); PEG-9 to PEG-50 palmitate (as the INCI names: PEG-9 palmitate to PEG-50 palmitate); PEG-9 to PEG-50 stearate (as the INCI names: PEG-9 stearate to PEG-50 stearate); PEG-9 to PEG-50 palmitostearate; PEG-9 to PEG-50 behenate (as the INCI names: PEG-9 behenate to PEG-50 behenate); polyethylene glycol 100 EO monostearate (INCI name: PEG-100 stearate); and mixtures thereof.

Nonlimiting examples of nonionic emulsifiers having a Hydrophile-Lipophile Balance (HLB) from about 9 to about 12 include alkylpolyglucosides (e.g., C12-20 alkyl glucoside), polyglycerol-based emulsifiers (e.g., polygyceryl-3 methylglucose distearate), sorbitan fatty esters, sugar fatty esters, polyol fatty esters, ethoxylates thereof, or a mixture thereof.

Nonlimiting examples of glyceryl esters include bis-diglyceryl polyacyladipate-2, glyceryl behenate, glyceryl caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl dioleate, glyceryl distearate, glyceryl laurate, or a mixture thereof. In at least one instance the glyceryl ester comprises glyceryl stearate, bis-diglyceryl polyacyladipate, glyceryl ricinoleate, and mixtures thereof.

Nonlimiting examples of fatty alcohols include fatty alcohols having from 12 to 24 carbon atoms, in particular, C14-24 alcohols, cetyl alcohol, stearyl alcohol, cetearyl alcohol, behenyl alcohol, lauryl alcohol, myristic or myristyl alcohol, arachidyl alcohol, lignoceryl alcohol, and mixtures thereof.

Nonlimiting examples of fatty compounds include fatty esters (e.g., diisopropyl sebacate, isononyl isononanoate), polyolefins (petrolatum), waxes, squalane, squalene, hydrogenated polyisobutene, hydrogenated polydecene, polybutene, mineral oil, pentahydrosqualene, plant and/or vegetable oil (e.g., soybean oil), hydrocarbon-based oils (isohexadecane), silicone oils (e.g., dimethicone, dimethiconol, and a mixture thereof.

Nonlimiting examples of thickening polymers include taurate copolymers, polyacrylates, polyacrylamides, etc., e.g., polyacrylate crosspolymer-6, sodium polyacrylate, polyacrylamide. Nonlimiting examples of taurate copolymers include acrylamide/sodium acryloyl dimethyl taurate copolymer, hydroxyethyl acrylate/sodium acryloyl dimethyl taurate copolymer, ammonium acryloyldimethyl taurate/VP copolymer, sodium acrylate/sodium acryloyl dimethyl taurate copolymer, hydroxyethyl acrylate/ sodium acryloyldimethyl taurate copolymer, and mixtures thereof.

Nonlimiting examples of water-soluble solvents include glycerin, C₂-C₅ mono-alcohols, polyols (polyhydric alcohols), glycols, and a mixture thereof. In an embodiment, the one or more water-soluble solvents may be chosen from propylene glycol, butylene glycol, pentylene glycole, dipropylene glycol, ethanol, isopropanol, t-butyl alcohol, and mixtures thereof.

In an embodiment, the cosmetic compositions may optionally include one or more miscellaneous ingredients. Nonlimiting examples of miscellaneous ingredients include miscellaneous emulsifiers/surfactants other than the nonionic emulsifiers of plurality of nonionic emulsifiers of (b), preservatives, fragrances, pH adjusters, salts, buffers, antioxidants, flavonoids, vitamins, botanical extracts, UV filtering agents, proteins, protein hydrolysates and/or isolates, hydrotropes, pearlescent agents, fillers, colorants, mattifying agents, further skin active agents, depigmenting agents, anti-wrinkle agents, and mixtures thereof. In an embodiment, the cosmetic composition includes at least one further skin active agent.

In an embodiment, the cosmetic compositions form part of a kit comprising a cosmetic composition according to the instant disclosure and one or more separately contained compositions. In an embodiment, the compositions are received in a device, for example, a device that dispenses the cosmetic composition and the one or more separately contained compositions. In an embodiment, the device mixes the cosmetic compositions with one or more additional cosmetic compositions before dispensing the mixture. In another embodiment, the device independently and optionally concurrently dispenses the cosmetic composition of the instant disclosure and the one or more additional cosmetic compositions (without mixing them prior to dispensing them). Even though high amounts of retinol are included in the cosmetic compositions, the compositions are unique in that they are compatible with other cosmetic compositions, in particular, other cosmetic compositions for treating the skin.

Another aspect of the instant disclosure relates to methods for treating skin. The methods include applying the cosmetic composition according to the instant disclosure to the skin. In an embodiment, the methods relate to boosting collagen and/or elastin production, reducing the appearance of fine lines, wrinkles, and uneven skin tone, tightening the skin, preventing/or treating acne, decreasing the development of melanin, and improving and brightening complexion.

Another aspect of the instant disclosure relates to methods for stabilizing cosmetic compositions containing high amounts of retinol. Another aspect of the instant disclosure is stabilizing and preserving high amounts of retinol in the cosmetic compositions. These methods, as describe throughout the disclosure, comprise incorporating the high amounts of retinol into the compositions of the instant disclosure.

Other features and iterations of the invention are described in more detail below.

### DETAILED DESCRIPTION

A common problem associated with formulating compositions, especially composition comprising multiple components, is ensuring physical stability, chemical stability, solubility, and the like. Many additives for food, cosmetics, personal care, and household products into which they are incorporated are difficult to stabilize and solubilize, especially when used in high amounts. The consequence of stability and solubility problems is significant. For example, stability problems can cause partial, if not complete, loss of product integrity, color loss, malodor, viscosity changes, etc. Stability and solubility problems can cause an increased or a decreased amount of the component in question to be applied. With respect to active ingredients, stability and solubility problems reduce or eliminate activity, and prevent the active ingredients from reaching their intended target in the desired amount.

With aging, the outer skin layer (epidermis) thins, even though the number of cell layers remains unchanged. The number of pigment-containing cells (melanocytes), however, decreases. Therefore, the skin appears pale and translucent. Large pigmented spots (age spots, liver spots, or lentigos) may appear in sun-exposed areas. Changes in the connective tissue reduce the skin’s strength and elasticity. This is known as elastosis. It is more noticeable in sun-exposed areas (solar elastosis). Elastosis produces the leathery, weather-beaten appearance common to farmers, sailors, and others who spend a large amount of time outdoors. Dehydration increases the risk of skin injury. Poor nutrition can also negatively influence the skin, causing dryness, rash, and puffiness.

Human skin acts as a primary barrier between the body and its environment. Crucial for this skin barrier function is the lipid matrix in the outermost layer of the skin (epidermis), the stratum corneum (SC). Two of its functions are (1) to prevent excessive water loss through the epidermis and (2) to avoid that compounds from the environment permeate into the viable epidermal and dermal layers and thereby provoke an immune response. The composition of the SC lipid matrix is dominated by three lipid classes: cholesterol, free fatty acids, and ceramides. These lipids adopt a highly ordered, 3-dimensional structure of stacked densely packed lipid layers (lipid lamellae): the lateral and lamellar lipid organization. The way in which these lipids are ordered depends on the composition of the lipids. One very common skin disease in which the SC lipid barrier is affected is atopic dermatitis (AD).

What is needed, among other things, are stable compositions which include and maintain high concentrations of retinol, that boost collagen and/or elastin production, reduce the appearance of fine lines, wrinkles, and uneven skin tone, tighten the skin, prevent/or treat acne, decrease the development of melanin, and improve and brighten complexion.

In an embodiment, the present disclosure is directed to, among other things, a cosmetic composition that includes high amounts of retinol; and to methods for stabilizing (preserving) the high amounts of retinol. In an embodiment, the compositions include:
(a) about 0.05 to about 6 wt.% of retinol;
(b) about 1 to about 8 wt.% of a plurality of nonionic emulsifiers comprising:
   (i) one or more ethoxylated fatty acids; and
   (ii) one or more nonionic emulsifiers having a Hydrophile-Lipophile Balance (HLB) from about 9 to about 12;
   (iii) optionally one or more glyceryl esters;
   (iv) optionally, one or more additional nonionic emulsifiers;
(c) about 0.1 to about 8 wt.% of one or more fatty alcohols;
(d) about 1 to about 20 wt.% of one or more fatty compounds;
(e) one or more thickening polymers; and
(f) water;
   wherein the composition is in the form of an oil in water emulsion and all percentages by weight are based on the total weight of the cosmetic composition.

### (A) Retinol

Retinol (also called vitamin A₁) is a vitamin in the vitamin A family. Retinol, among other things, boosts skin’s surface renewal process and helps to strengthen skin’s foundation to minimize the look of age spots, improve skin texture, and help resist the look of lines and wrinkles.

The amount of retinol will vary. Nonetheless, in various embodiments, the amount of retinol in the cosmetic compositions is from about 0.05 to about 6 wt.%, based on the total weight of the composition. In further embodiments, the amount of retinol in the composition is from about 0.05 to about 5 wt.%, about 0.05 to about 4 wt.%, about 0.05 to about 3 wt.%, about 0.05 to about 2 wt.%, about 0.1 to about 6 wt.%, about 0.1 to about 5 wt.%, about 0.1 to about 4 wt.%, about 0.1 to about 3 wt.%, about 0.1 to about 2 wt.%, about 0.5 to about 5 wt.%, about 0.5 to about 4 wt.%, about 0.5 to about 3 wt.%, or about 0.5 to about 2 wt.%, based on the total weight of the cosmetic composition.

### (B) Plurality of Nonionic Emulsifiers

The cosmetic compositions may include a plurality of nonionic emulsifiers. In an embodiment, the cosmetic compositions include a plurality of nonionic emulsifiers comprising or consisting of: (i) one or more ethoxylated fatty acids; and (ii) one or more nonionic emulsifiers having a Hydrophile-Lipophile Balance (HLB) from about 9 to about 12; (iii) optionally, (iii) one or more glyceryl esters; and (iv) optionally, one or more additional nonionic emulsifiers. The plurality of nonionic emulsifiers represents all the nonionic emulsifiers in the cosmetic compositions.

The total amount of the plurality of nonionic emulsifiers (all nonionic emulsifiers) in the cosmetic compositions will vary. Nonetheless, in various embodiments, the total amount of the plurality of nonionic emulsifiers is from about 1 to about 8 wt.%, based on the total weight of the cosmetic composition. In further embodiments, the total amount of the plurality of nonionic emulsifiers is from about 1 to about 6 wt.%, about 1 to about 5 wt.%, about 1 to about 4 wt.%, about 1 to about 3 wt.%, about 1.1 to about 8 wt.%, about 1.1 to about 6 wt.%, about 1.1 to about 5 wt.%, about 1.1 to about 4 wt.%, about 1.1 to about 3 wt.%, about 1.1 to about 2 wt.%, about 1.5 to about 8 wt.%, about 1.5 to about 6 wt.%, about 1.5 to about 5 wt.%, about 1.5 to about 4 wt.%, or about 1.5 to about 3 wt.%, 2 to about 8 wt.%, about 2 to about 6 wt.%, about 2 to about 5 wt.%, about 2 to about 4 wt.%, or about 2 to about 3 wt.%, based on the total weight of the cosmetic composition.

### (B)(i) Ethoxylated Fatty Acids

Ethoxylated fatty acids are also known as “fatty acid ethoxylates,” which are produced by the process of ethoxylation on fatty acids. These are nonionic surface-active agents manufactured by addition of ethylene oxide to fatty acids producing a range of ethoxylates having different moles of EO. Ethoxylated fatty acids range from clear liquid, pasty, or waxy solids. This nature depends on the length of alkyl chain and the apparent number of ethoxyl groups. Since ethoxylated fatty acids have various HLB values based on their moles of EO.

Nonlimiting examples of ethoxylated fatty acids include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the INCI names: PEG-9 laurate to PEG-50 laurate); PEG-9 to PEG-50 palmitate (as the INCI names: PEG-9 palmitate to PEG-50 palmitate); PEG-9 to PEG-50 stearate (as the INCI names: PEG-9 stearate to PEG-50 stearate); PEG-9 to PEG-50 palmitostearate; PEG-9 to PEG-50 behenate (as the INCI names: PEG-9 behenate to PEG-50 behenate); polyethylene glycol 100 EO monostearate (INCI name: PEG-100 stearate); and mixtures thereof.

In an embodiments, the one or more ethoxylated fatty acids are esters with fatty acids of chain length C12-C40 and a degree of ethoxylation up to 100, preferably from 5-100, for example, PEG-20 stearate, PEG-30 stearate, PEG-40 stearate, PEG-100 stearate, and mixtures thereof.

The total amount of the one or more ethoxylated fatty acids may be from about 0.1 to about 5 wt.%, based on the total weight of the cosmetic composition. In various embodiments, the total amount of the one or more ethoxylated fatty acids may be from about 0.1 to about 4 wt.%, about 0.1 to about 3 wt.%, about 0.1 to about 2 wt.%, about 0.2 to about 5 wt.%, about 0.2 to about 4 wt.%, about 0.2 to about 3 wt.%, or about 0.2 to about 2 wt.%, about 0.4 to about 5 wt.%, about 0.4 to about 4 wt.%, about 0.4 to about 3 wt.%, or about 0.4 to about 2 wt.%, based on the total weight of the cosmetic composition.

### (B)(ii) Nonionic Emulsifiers having an HLB From 9 to 12

Nonlimiting examples of nonionic emulsifiers having an HLB from about 9 to about 12 include:
(1) Polyglyceryl fatty acid esters, polyoxyalkylenated alkyl glycerides, and polyoxyalkylenated fatty ethers, preferably, polyglyceryl fatty acid esters, polyoxyalkylenated alkyl glycerides, and polyoxyalkylenated fatty ethers that are solid at 25° C. ;
(2) Mixed esters of fatty acid or of fatty alcohol, of carboxylic acid and of glycerol;
(3) Fatty acid esters of sugars and fatty alcohol ethers of sugars; and
(4) Fatty esters of sorbitan and oxyalkylenated fatty esters of sorbitan;

In various embodiments, the nonionic emulsifiers having an HLB from about 9 to about 12 may be chosen from: polyglyceryl fatty acid esters of at least one, preferably one, fatty acid comprising at least one saturated or unsaturated, linear or branched C₈-C₂₂ hydrocarbon group such as C₈-C₂₂ alkyl or alkenyl group, preferably C₈-C₁₈ alkyl or alkenyl group, and more preferably C₈-C₁₂ alkyl or alkenyl group, and of 2 to 12 glycerols, preferably 2 to 10 glycerols and more preferably 2 to 8 glycerols; polyoxyethylenated alkyl glycerides such as polyethylene glycol derivatives of a mixture of mono-, di- and tri-glycerides of caprylic and capric acids (preferably 2 to 30 ethylene oxide units, more preferably 2 to 20 ethylene oxide units, and even more preferably 2 to 10 ethylene oxide units); polyoxyethylenated fatty ethers of at least one, preferably one, fatty alcohol comprising at least one saturated or unsaturated, linear or branched C₈-C₂₂ hydrocarbon group such as C₈-C₂₂ alkyl or alkenyl group, preferably C₈-C₁₈ alkyl or alkenyl group, and more preferably C₈-C₁₂ alkyl or alkenyl group, and of 2 to 60 ethylene oxides, preferably from 2 to 30 ethylene oxides, and more preferably from 2 to 10 ethylene oxides; and mixtures thereof.

In an embodiment, it is preferable that the polyglyceryl fatty acid ester have a polyglycerol moiety derived from 2 to 10 glycerols, more preferably from 2 to 8 glycerols, and further more preferably 4 to 6 glycerols. The polyglyceryl fatty acid ester may be chosen from the mono, di and tri esters of saturated or unsaturated acid, preferably saturated acid, including 8 to 22 carbon atoms, preferably 8 to 18 carbon atoms, and more preferably 8 to 12 carbon atoms, such as caprylic acid, capric acid, lauric acid, oleic acid, stearic acid, isostearic acid, and myristic acid.

The polyoxyalkylenated fatty ethers, preferably polyoxyethylenated fatty ethers, may comprise from 2 to 60 ethylene oxide units, preferably from 2 to 30 ethylene oxide units, and more preferably from 2 to 10 ethylene oxide units. The fatty chain of the ethers may be chosen in particular from lauryl, behenyl, arachidyl, stearyl and cetyl units, and mixtures thereof, such as cetearyl. Examples of ethoxylated fatty ethers which may be mentioned are lauryl alcohol ethers comprising 2, 3, 4, and 5 ethylene oxide units (CTFA names: Laureth-2, Laureth-3, Laureth-4, and Laureth-5).

The mixed esters of fatty acids, or of fatty alcohol, of carboxylic acid and of glycerol, which can be used as the above nonionic surfactant, may be chosen in particular from the group comprising mixed esters of fatty acid or of fatty alcohol with an alkyl or alkenyl chain containing from 12 to 22 carbon atoms, preferably from 8 to 18 carbon atoms, and more preferably from 12 to 12 carbon atoms, and of alpha-hydroxy acid and/or of succinic acid, with glycerol. The alpha-hydroxy acid may be, for example, citric acid, lactic acid, glycolic acid or malic acid, and mixtures thereof.

The alkyl chain of the fatty acids or alcohols from which are derived the mixed esters which can be used may be linear or branched, and preferably saturated. They may especially be stearate, isostearate, behenate, arachidonate, palmitate, myristate, laurate, isostearyl, stearyl, behenyl, myristyl, or lauryl chains, and mixtures thereof. nonlimiting examples of mixed esters include the mixed ester of glycerol and of the mixture of citric acid, lactic acid, linoleic acid and oleic acid (CTFA name: Glyceryl citrate/lactate/linoleate/oleate); the mixed ester of succinic acid and of isostearyl alcohol with glycerol (CTFA name: Isostearyl diglyceryl succinate); the mixed ester of citric acid and of stearic acid with glycerol (CTFA name: Glyceryl stearate citrate); the mixed ester of lactic acid and of stearic acid with glycerol (CTFA name: Glyceryl stearate lactate), and mixtures thereof.

The fatty acid esters of sugars may be chosen in particular from the group comprising esters or mixtures of esters of C₁₂-C₂₂ fatty acid and of sucrose, of maltose, of glucose or of fructose, and esters or mixtures of esters of C₁₄-C₂₂ fatty acid and of methylglucose.

The C₁₂-C₂₂ or C₁₄-C₂₂ fatty acids forming the fatty unit of the esters which can be used comprise a saturated or unsaturated linear alkyl or alkenyl chain containing, respectively, from 12 to 22 or from 14 to 22 carbon atoms. The fatty unit of the esters may be chosen in particular from stearates, behenates, arachidonates, palmitates, myristates, laurates and caprates, and mixtures thereof. Stearates are preferably used.

In an embodiment, the cosmetic composition preferably includes at least one ester of C₁₄-C₂₂ fatty acids and of methylglucose, for example, polyglyceryl-3 methylglucose distearate.

As nonlimiting examples of esters or mixtures of esters of fatty acid and of sucrose, of maltose, of glucose or of fructose, mention may be made of sucrose monostearate, sucrose distearate and sucrose tristearate and mixtures thereof; and examples of esters or mixtures of esters of fatty acid and of methylglucose which may be mentioned are polyglyceryl-3 methylglucose distearate. Mention may also be made of glucose or maltose monoesters such as methyl o-hexadecanoyl-6-D-glucoside and o-hexadecanoyl-6-D-maltoside.

Nonlimiting examples of useful fatty alcohol ethers of sugars are solid at a temperature of less than or equal to 45° C., and in some embodiments, less than or equal to 25° C., and may be chosen in particular from the group comprising ethers or mixtures of ethers of C₈-C₂₂ fatty alcohol and of glucose, of maltose, of sucrose or of fructose, and ethers or mixtures of ethers of a C₁₄-C₂₂ fatty alcohol and of methylglucose. These are in particular, alkylpolyglucosides, which in various embodiments are preferred. A particularly preferred examples is C12-20 alkyl glucoside. Accordingly, in an embodiment, the cosmetic compositions include at least one alkylpolyglucosides, for example, C12-20 alkyl glucoside.

The C₁₂-C₂₂ or C₁₄-C₂₂ fatty alcohols forming the fatty unit of the ethers which may be used comprise a saturated or unsaturated, linear alkyl or alkenyl chain containing, respectively, from 12 to 22 or from 14 to 22 carbon atoms. The fatty unit of the ethers may be chosen in particular from decyl, cetyl, behenyl, arachidyl, stearyl, palmityl, myristyl, lauryl, and hexadecanoyl units, and mixtures thereof, such as cetearyl.

As nonlimiting examples of fatty alcohol ethers of sugars, mention may be made of alkylpolyglucosides such as laurylglucoside, cetostearyl glucoside, arachidyl glucoside, and mixtures thereof. Further mention is made of sucrose monostearate, sucrose distearate or sucrose tristearate and mixtures thereof, polyglyceryl-3 methylglucose distearate and alkylpolyglucosides.

The fatty esters of sorbitan and oxyalkylenated fatty esters of sorbitan may, in various embodiments, be chosen from the group comprising C₁₆-C₂₂ fatty acid esters of sorbitan and oxyethylenated C₁₆-C₂₂ fatty acid esters of sorbitan. They may be formed from at least one fatty acid comprising at least one saturated linear alkyl chain containing, respectively, from 16 to 22 carbon atoms, and from sorbitol or from ethoxylated sorbitol. The oxyethylenated esters may generally comprise from 1 to 100 ethylene glycol units and preferably from 2 to 40 ethylene oxide (EO) units. These esters may be chosen in particular from stearates, behenates, arachidates, palmitates, and mixtures thereof. Stearates and palmitates are preferably used. Nonlimiting examples include sorbitan monostearate (CTFA name: sorbitan stearate), sorbitan monopalmitate (CTFA name: sorbitan palmitate), sorbitan tristearate 20 EO (CTFA name: polysorbate 65), and mixtures thereof.

In an embodiment, the one or more nonionic emulsifiers having an HLB from 9 to 12 may be chosen from alkylpolyglucosides (e.g., C12-20 alkyl glucoside), polyglyceryl-10 laurate, polyglyceryl-2 dipolyhydroxystearate, polyglyceryl-2 polyhydroxystearate, polyglyceryl-3 methylglucose distearate, polyglyceryl-3 polyricinoleate, polyglyceryl-3 ricinoleate, and a mixture thereof.

The total amount of the one or more nonionic emulsifiers having an HLB from 9 to 12 will vary. Nonetheless, in various embodiment, the total amount of the one or more nonionic emulsifiers having an HLB from 9 to 12 is from about 0.1 to about 5 wt.%, based on the total weight of the cosmetic composition. In further embodiments, the total amount of the one or more nonionic emulsifiers having an HLB from 9 to 12 is from about 0.1 to about 4 wt.%, about 0.1 to about 3 wt.%, about 0.1 to about 2 wt.%, about 0.2 to about 5 wt.%, about 0.2 to about 4 wt.%, about 0.2 to about 3 wt.%, or about 0.2 to about 2 wt.%, about 0.3 to about 5 wt.%, about 0.3 to about 4 wt.%, about 0.3 to about 3 wt.%, or about 0.3 to about 2 wt.%, about 0.5 to about 5 wt.%, about 0.5 to about 4 wt.%, about 0.5 to about 3 wt.%, or about 0.5 to about 2 wt.%, about 1 to about 5 wt.%, about 1 to about 4 wt.%, about 1 to about 3 wt.%, or about 1 to about 2 wt.%, based on the total weight of the cosmetic composition.

### (B)(iii) Glyceryl Fatty Esters

Glyceryl esters are also known as “glyceryl fatty esters.” Non-limiting examples include: glyceryl myristate, glyceryl capromyristate, glyceryl stearate, glyceryl hydroxy stearate, glyceryl isostearate, glyceryl ricinoleate, glyceryl dilaurate, glyceryldistearate, glycerylmono/dimyristate, glycerylstearatepalmitate, caprylic/capricdiglycerylsuccinate, caprylic/capric/stearictriglycerides, glyceryltrilaurate/stearate, glyceryldi/tripalmitostearate, glyceryldi/tritristearate, caprylic/capric/laurictriglycerides, glyceryltriheptanoate, glyceryl trioctanoate, glyceryl trilaurate, glyceryltristearate, glyceryltris-12-hydroxystearate, glyceryltriacetyl hydroxystearate, glyceryl triacetyl ricinioleate, glyceryl triisostearate, glyceryl tribehenate, and mixtures thereof.

In various embodiments, the one or more glyceryl esters may include glyceryl monoesters, such as glyceryl monoesters of C16-C22. Preferably saturated and branched chain fatty acids such as glyceryl monostearate, glyceryl monoisostearate, glyceryl monopalmitate, glyceryl monobehenate, and mixtures thereof. In a preferred embodiment, the glyceryl ester may be chosen from glyceryl monostearate (glyceryl stearate), glyceryl monoisostearate, glyceryl monopalmitate, glyceryl monobehenate, and mixtures thereof. In one embodiment, the glyceryl ester is glyceryl oleate. Nonlimiting examples of nonionic emulsifiers that are glyceryl esters include glyceryl behenate, glyceryl erucate, glyceryl hydroxystearate, glyceryl isostearate, glyceryl laurate, glyceryl myristate, glyceryl palmitate lactate, glyceryl stearate, glyceryl distearate, glyceryl laurate, or a mixture thereof. preferred glyceryl esters include glyceryl stearate, glyceryl ricinoleate, or a mixture thereof.

In an embodiment, the one or more glyceryl esters are chosen from glyceryl myristate, glyceryl capromyristate, glyceryl stearate, glyceryl hydroxy stearate, glyceryl isostearate, glyceryl ricinoleate, glyceryl dilaurate, glyceryldistearate, and mixture thereof.

The total amount of the one or more glyceryl esters, if present, will vary. Nonetheless, in various embodiments, the total amount of the one or more glyceryl esters, when present, is from about 0.1 to about 5 wt.%, based on the total weight of the cosmetic composition. In further embodiments, the total amount of the one or more glyceryls esters is from about 0.1 to about 4 wt.%, about 0.1 to about 3 wt.%, about 0.1 to about 2 wt.%, about 0.2 to about 5 wt.%, about 0.2 to about 4 wt.%, about 0.2 to about 3 wt.%, or about 0.2 to about 2 wt.%, about 0.3 to about 5 wt.%, about 0.3 to about 4 wt.%, about 0.3 to about 3 wt.%, or about 0.3 to about 2 wt.% based on the total weight of the cosmetic composition.

### (B)(iv) Additional Nonionic Emulsifiers

Nonionic emulsifiers that are not ethoxylated fatty acids (b)(i), not nonionic emulsifiers having an HLB from about 9 to about 12 (b)(ii) and are not glyceryl fatty esters (b)(iii) are classified as “additional nonionic emulsifiers.” The cosmetic compositions of the instant disclosure may optionally include one or more additional nonionic emulsifiers.

In an embodiment, the one or more additional nonionic emulsifiers include one or more nonionic emulsifiers having an HLB of greater than or equal to about 14. Nonlimiting examples include polysorbate 60 (HLB=14.9), polysorbate 80 (HLB=15), isosteareth-20 (HLB=15), PEG-60 almond glycerides (HLB=15), polysorbate 80 (HLB=15), PEG-20 methyl glucose sesquistearate (HLB=15), ceteareth-20 (HLB=15.2), oleth-20 (HLB=15.3), steareth-20 (HLB=15.3), steareth-21 (HLB=15.5), ceteth-20 (HLB=15.7), isoceteth-20 (HLB=15.7), polysorbate 20 (HLB=16.7), polysorbate 20 (HLB=16.7), laureth-23 (HLB=16.9), PEG-100 stearate (HLB=18.8), steareth-100 (HLB=18.8), PEG-80 sorbitan laurate (HLB=19.1), and mixtures thereof.

In an embodiment, the one or more additional nonionic emulsifiers includes one or more nonionic emulsifiers having an HLB from about 1 to about 5. Nonlimiting examples include sorbitan trioleate (HLB=1.8), sorbitan sesquioleate (HLB=3.7), lecithin (HLB=4), sorbitan oleate (HLB=4.3), sorbitan monostearate (HLB=4.7), sorbitan stearate (HLB=4.7), sorbitan isostearate (HLB=4.7), steareth-2 (HLB=4.9), oleth-2 (HLB=4.9), and mixtures thereof.

The total amount of the one or more additional nonionic emulsifiers, if present, will vary. Nonetheless, in various embodiments, the total amount of the one or more additional nonionic emulsifiers, when present, is from about 0.01 to about 5 wt.%, based on the total weight of the cosmetic composition. In further embodiments, the total amount of the one or more additional nonionic emulsifiers is in an amount of about 0.01 to about 4 wt.%, about 0.01 to about 3 wt.%, about 0.01 to about 2 wt.%, about 0.01 to about 1 wt.%, about 0.01 to about 0.5 wt.%, about 0.1 to about 5 wt.%, about 0.1 to about 4 wt.%, about 0.1 to about 3 wt.%, about 0.1 to about 2 wt.%, about 0.1 to about 1 wt.%, based on the total weight of the cosmetic composition.

### (C) Fatty Alcohols

The term “fatty alcohol” means an alcohol comprising at least one hydroxyl group (OH), and comprising at least 8 carbon atoms, and which is neither oxyalkylenated (in particular neither oxyethylenated nor oxypropylenated) nor glycerolated. The fatty alcohols can be represented by: R-OH, wherein R denotes a saturated (alkyl) or unsaturated (alkenyl) group, linear or branched, comprising from 8 to 40 carbon atoms, preferably 10 to 30 carbon atoms, more preferably 12 to 24 carbon atoms, and even more preferably 14 to 22 carbon atoms.

The fatty alcohol(s) may be liquid or solid. In some instances, it is preferable that the cosmetic compositions include at least one solid fatty alcohol. The solid fatty alcohols that can be used include those that are solid at ambient temperature and at atmospheric pressure (25° C., 780 mmHg), and are insoluble in water, that is to say they have a water solubility of less than 1% by weight, preferably less than 0.5% by weight, at 25° C., 1 atm.

The solid fatty alcohols may be represented by: R-OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

Non-limiting examples of useful fatty alcohols include lauryl alcohol or lauryl alcohol (1-dodecanol); myristic or myristyl alcohol (1-tetradecanol); cetyl alcohol (1-hexadecanol); stearyl alcohol (1-octadecanol); arachidyl alcohol (1-eicosanol); behenyl alcohol (1-docosanol); lignoceryl alcohol (1-tetracosanol); ceryl alcohol (1-hexacosanol); montanyl alcohol (1-octacosanol); myricylic alcohol (1-triacontanol), and mixtures thereof.

In certain embodiments, the one or more fatty alcohols have from 12 to 24 carbon atoms. Specific nonlimiting examples include C14-22 alcohols, cetyl alcohol, stearyl alcohol, cetearyl alcohol, behenyl alcohol, lauryl alcohol, myristic or myristyl alcohol, arachidyl alcohol, lignoceryl alcohol, or mixtures thereof.

The fatty alcohol(s) may be liquid or solid. However, in certain embodiments, it is preferable that the cosmetic compositions include at least one solid fatty alcohol, in particular saturated fatty alcohols that are solid at 25° C., preferably having at least 12 carbon atoms. Preferably, the cosmetic composition includes one or more solid fatty alcohols, for example, chosen from C14-22 alcohols, cetyl alcohol, stearyl alcohol, behenyl alcohol and mixtures thereof, preferably cetyl alcohol, behenyl alcohol, cetearyl alcohol, and mixtures thereof.

In some instances, the cosmetic compositions include one or more fatty alcohols selected from C14-22 alcohols, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a mixture thereof. In some instances, the cosmetic compositions preferably include cetyl alcohol, behenyl alcohol, and cetearyl alcohol.

The total amount of the one or more fatty alcohols in the cosmetic compositions will vary. Nonetheless, in various embodiments, the total amount of the one or more fatty alcohols is from 0.1 wt.% to about 8 wt.%, based on the total weight of the composition. In further embodiments, the total amount of one or more fatty alcohols in the composition is from about 0.1 to about 6 wt.%, about 0.1 to about 5 wt.%, about 0.1 to about 4 wt.%, about 0.5 to about 8 wt.%, about 0.5 to about 6 wt.%, about 0.5 to about 5 wt.%, about 0.5 to about 4 wt.%, about 1 to about 8 wt.%, about 1 to about 6 wt.%, about 1 to about 5 wt.%, about 1 to about 4 wt.%, or about 1 to about 3 wt.%, based on the total weight of the composition.

### (D) Fatty Compounds

The term “fatty compounds” is interchangeable with the “fatty materials.” Fatty compounds are known as compounds that are not soluble (or only sparingly soluble) in water; they are hydrophilic and are often solubilized in organic solvents. They include materials such as oils, fats, waxes, hydrocarbons, fatty esters, etc. For purposes of the instant disclosure, “fatty compounds” do not include fatty acids, which are separately referred to above. However, for purposes of the instant disclosure, “silicone oils” are considered fatty compounds. Non-limiting examples of useful fatty compounds include oils, waxes, alkanes (paraffins), fatty acids, fatty esters, triglyceride compounds, lanolin, hydrocarbons, derivatives thereof, and mixtures thereof. Fatty compounds are described by the International Federation Societies of Cosmetic Chemists, for example, in Cosmetic Raw Material Analysis and Quality, Volume I: Hydrocarbons, Glycerides, Waxes and Other Esters (Redwood Books, 1994), which is incorporated herein by reference in its entirety.

Non-limiting examples of fatty compounds include oils, mineral oil, alkanes (paraffins), fatty acids, fatty alcohol derivatives, fatty acid derivatives, esters of fatty alcohols, hydroxy-substituted fatty acids, waxes, triglyceride compounds, lanolin, silicone oils, and mixtures thereof.

### Fatty Acids

In some instances, the fatty compounds include fatty acids, fatty acid derivatives, esters of fatty acids, and hydroxyl-substituted fatty acids. The fatty acids may be straight or branched chain acids and/or may be saturated or unsaturated. Non-limiting examples of fatty acids include diacids, triacids, and other multiple acids as well as salts of these fatty acids. For example, the fatty acid may optionally include or be chosen from lauric acid, palmitic acid, stearic acid, behenic acid, arichidonic acid, oleic acid, isostearic acid, sebacic acid, and a mixture thereof. In some cases, the fatty acids are selected from the group consisting of palmitic acid, stearic acid, and a mixture thereof.

### Waxes

The fatty compounds may, in some instances, include or be chosen from one or more waxes. Non-limiting examples of waxes in this category include for example, synthetic wax, ceresin, paraffin, ozokerite, polyethylene waxes, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, acacia decurrents flower wax, vegetable waxes (such as sunflower seed (helianthus annuus), carnauba, candelilla, ouricury or japan wax or cork fibre or sugarcane waxes), or a mixture thereof.

### Non-Silicone Oils

In some instances, the fatty compounds may include or be chosen from one or more oil(s). Suitable oils include, but are not limited to, natural oils, such as coconut oil; hydrocarbons, such as mineral oil and hydrogenated polyisobutene; fatty alcohols, such as octyldodecanol; esters, such as C₁₂-C₁₅ alkyl benzoate; diesters, such as propylene dipelarganate; and triesters, such as glyceryl trioctanoate. Non-limiting examples of oils that may, optionally, be included in the cosmetic compositions include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, castor oil, lanolin and lanolin derivatives, triisocetyl citrate, sorbitan sesquioleate, C₁₀-C₁₈triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or combinations thereof.

### Silicone Oils

Nonlimiting examples of silicone oils include dimethicone, dimethiconol, dimethiconol, cyclomethicone, polysilicone-11, phenyl trimethicone, trimethylsilylamodimethicone, and stearoxytrimethylsilane. In a preferred embodiment, the one or more silicones oils are non-volatile silicon oils. Useful silicone oils include polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates. Other examples of silicone oils that may be mentioned include volatile linear or cyclic silicones, such as those with a viscosity 8 centistokes (8x106 m2/s) and/or containing from 2 to 7 silicon atoms. These silicones optionally comprise alkyl or alkoxy groups containing from 1 to 10 carbon atoms. Non-limiting examples of volatile silicone oils include octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, or mixtures thereof.

In a preferred embodiment, the cosmetic compositions include one or more silicone oils chosen from dimethicone, dimethiconol, cyclomethicone, polysilicone-11, phenyl trimethicone, and amodimethicone, preferably dimethicone.

In an embodiment, cosmetic compositions include one or more amino functionalized silicones. Nonlimiting examples include amodimethicone, bis-hydroxy/methoxy amodimethicones, bis-cetearyl amodimethicone, amodimethicone, bis(C13-15 alkoxy) PG amodimethicones, aminopropyl phenyl trimethicones, aminopropyl dimethicones, bis-amino PEG/PPG-41/3 aminoethyl PG-propyl dimethicones, caprylyl methicones, and a mixture thereof. Amodimethicone is a particularly useful amino functionalized silicone.

In a preferred embodiment the one or more silicone oils are chosen from dimethicone, dimethiconol, cyclopentasiloxane, cyclomethicone, cyclotetrasiloxane, cyclohexasiloxane, cycloheptasiloxane, decamethylcyclopentasiloxane, cyclotetrasiloxane, cyclotrisiloxane, capryldimethicone, caprylyl trimethicone, caprylyl methicone, cetearylmethicone, hexadecylmethicone, hexylmethicone, lauryl methicone, myristyl methicone, phenyl methicone, stearyl methicone, stearyl dimethicone, behenyl dimethicone, trifluoropropyl methicone, cetyl dimethicone, polyphenylmethylsiloxane, dimethylpolysiloxane, methylphenylpolysiloxane, methyltrimethicone, diphenylsiloxyphenyl trimethicone, and phenyl trimethicone, and mixtures thereof. Preferably, the one or more silicone oils include dimethicone, and optionally one or more further silicone oils.

In some embodiments, the cosmetic composition may include one or more fatty compounds chosen from fatty esters (such as isononyl isononanoate), polyolefins (such as petrolatum), waxes, squalane, squalene, hydrogenated polyisobutene, hydrogenated polydecene, polybutene, mineral oil, pentahydrosqualene, plant and/or vegetable oil, hydrocarbon-based oils (such as isohexadecane), silicone oils (such as dimethicone), or a mixture thereof.

The amount of the one or more fatty compounds in the cosmetic compositions will vary. Nonetheless, in various embodiments, the total amount of the one or more fatty compounds is from about 1 wt.% to about 20 wt.%, based on the total weight of the cosmetic composition. In further embodiments, the amount of the one or more fatty compounds in the cosmetic compositions is from about 1 to 18 wt.%, about 1 to about 15 wt.%, about 5 to about 20 wt.%, about 5 to about 20 wt.%, about 5 to about 15 wt.%, about 6 to about 20 wt.%, about 6 to about 18 wt.%, about 6 to about 15 wt.%, about 8 to about 20 wt.%, about 8 to about 18 wt.%, or about 8 to about 15 wt.%, based on the total weight of the cosmetic composition.

### (E) Thickening Polymer

Non-limiting examples of thickening polymers include taurate copolymer, polyacrylate, polymethacrylate, polyethylacrylate, polyacrylamide, poly C10-30 alkyl acrylate, acrylic acid/acrylonitrogens copolymer, acrylates/steareth-20 itaconate copolymer, acrylates/ceteth-20 itaconate copolymer, Acrylates/Aminoacrylates/C10-30 Alkyl PEG-20 Itaconate Copolymer, acrylates/aminoacrylates copolymer, acrylates/steareth-20 methacrylate copolymer, acrylates/beheneth-25 methacrylate copolymer, acrylates/steareth-20 methacrylate crosspolymer, acrylates/beheneth-25 methacrylate/HEMA crosspolymer, acrylates/vinyl neodecanoate crosspolymer, acrylates/vinyl isodecanoate crosspolymer, Acrylates/Palmeth-25 Acrylate Copolymer, Acrylic Acid/Acrylamidomethyl Propane Sulfonic Acid Copolymer, and acrylates/C10-C30 alkyl acrylate crosspolymer, carbomers, hydrophobically modified polypolyacrylates; hydrophobically modified polyacrylic acids, hydrophobically modified polyacrylamides; hydrophobically modified polyethers wherein these materials may have a hydrophobe that can be selected from cetyl, stearyl, oleayl, and combinations thereof, acrylamide/ammonium acrylate copolymer, acrylates copolymer, Acrylates Crosspolymer-4, Acrylates Crosspolymer-3, acrylates/beheneth-25 methacrylate copolymer, acrylates/C10-C30 alkyl acrylate crosspolymer, acrylates/steareth-20 itaconate copolymer, ammonium polyacrylate/Isohexadecane/PEG-40 castor oil; sodium carbomer, crosslinked polyvinylpyrrolidone (PVP), polyacrylamide/C13-14 isoparaffin/laureth-7, polyacrylate 13/polyisobutene/polysorbate 20, polyacrylate crosspolymer-6, polyamide-3, polyquaternium-37, sodium polyacrylate, and a mixture thereof.

Among the nonionic thickening polymers that may be mentioned are:
(1) Celluloses modified with groups comprising at least one fatty chain; examples that may be mentioned include: hydroxyethylcelluloses modified with groups comprising at least one fatty chain, such as alkyl, arylalkyl or alkylaryl groups, or mixtures thereof, and in which the alkyl groups are preferably C8-C22, for instance the product NATROSOL PLUS GRADE 330 CS (C₁₆ alkyls) sold by the company Aqualon, or the product BERMOCOLL EHM 100 sold by the company Berol Nobel; and hydroxyethylcelluloses modified with alkylphenyl polyalkylene glycol ether groups, such as the product AMERCELL POLYMER HM-1500 (polyethylene glycol (15) nonylphenyl ether) sold by the company Amerchol,
(2) Hydroxypropyl guars modified with groups comprising at least one fatty chain, such as the product ESAFLOR HM 2(C₂₂ alkyl chain) sold by the company Lamberti, and the products RE210-18 (C₁₄ alkyl chain) and RE205-1 (C₂₀ alkyl chain) sold by the company Rhone-Poulenc,
(3) Copolymers of vinylpyrrolidone and of fatty-chain hydrophobic monomers; examples that may be mentioned include: the products ANTARON V216 or GANEX V216 (vinylpyrrolidone/hexadecene copolymer) sold by the company I.S.P. the products ANTARON V220 or GANEX V220 (vinylpyrrolidone/eicosene copolymer) sold by the company I.S.P.,
(4) Copolymers of C₁-C₆ alkyl methacrylates or acrylates and of amphiphilic monomers comprising at least one fatty chain, for instance the oxyethylenated methyl acrylate/stearyl acrylate copolymer sold by the company Goldschmidt under the name ANTIL 208,
(5) Copolymers of hydrophilic methacrylates or acrylates and of hydrophobic monomers comprising at least one fatty chain, for instance the polyethylene glycol methacrylate/lauryl methacrylate copolymer,
(6) Polyurethane polyethers comprising in their chain both hydrophilic blocks usually of polyoxyethylenated nature and hydrophobic blocks, which may be aliphatic sequences alone and/or cycloaliphatic and/or aromatic sequences.

In a preferred embodiment, at least one of the one or more thickening polymers in the cosmetic composition is a taurate copolymer.

Nonlimiting examples taurate copolymers include acrylamide/sodium acryloyl dimethyl taurate copolymer, hydroxyethyl acrylate/sodium acryloyl dimethyl taurate copolymer, ammonium acryloyldimethyl taurate/VP copolymer, sodium acrylate/sodium acryloyl dimethyl taurate copolymer, hydroxyethyl acrylate/ sodium acryloyldimethyl taurate copolymer, and a mixture thereof

The taurate copolymers may be hydrophilic and may contain an acrylate component. The at least one taurate copolymer may include, for example, acrylamide/sodium acryloyl dimethyl taurate copolymer, hydroxyethyl acrylate/sodium acryloyl dimethyl taurate copolymer, and/or sodium acrylate/sodium acryloyl dimethyl taurate copolymer. In some instances, at least one taurate copolymer is obtainable from ethylenically unsaturated, sulpho-functional monomers and ethylenically unsaturated hydrophilic monomers, for example from crosslinked anionic copolymers of acrylamide or methacrylamide and of 2-acrylamido-2-methyl-propanesulfonic acid.

In some instances, the one or more taurate copolymers may be chosen from acrylamide/sodium acryloyl dimethyl taurate copolymer, hydroxyethyl acrylate/sodium acryloyl dimethyl taurate copolymer, ammonium acryloyldimethyl taurate/VP copolymer, and mixtures thereof. Furthermore, in some instances, the cosmetic compositions may include both hydroxyethyl acrylate/sodium acryloyl dimethyl taurate copolymer and ammonium acryloyldimethyl taurate/VP copolymer, and optionally further include poly C10-30 alkyl acrylate.

The amount of the one or more thickening polymers will vary depending on the type of thickening polymers used; and depending on the desired viscosity of the cosmetic composition. Therefore, in an embodiment, the total amount of the one or more thickening agents is sufficient to achieve the viscosities set forth throughout the instant disclosure. Nonetheless, in various embodiments, the total amount of the one or more thickening agents is from about 0.1 to about 8 wt.%, based on the total weight of the cosmetic composition. In further embodiments, the total amount of the one or more thickening agents is from about 0.1 to about 6 wt.%, about 0.1 to about 5 wt.%, about 0.1 to about 4 wt.%, about 0.1 to about 3 wt.%, about 0.5 to about 8 wt.%, about 0.5 to about 6 wt.%, about 0.5 to about 5 wt.%, about 0.5 to about 4 wt.%, about 0.5 to about 3 wt.%, about 1 to about 8 wt.%, about 1 to about 6 wt.%, about 1 to about 5 wt.%, about 1 to about 4 wt.%, about 1 to about 3 wt.%, or about 1 to about 2 wt.%, based on the total weight of the cosmetic composition.

### (F) Water

The amount of water in the cosmetic compositions can and will vary depending on the amount of the other components in the cosmetic compositions. In various embodiments, the total amount of water in the compositions is from about 55 to about 85 wt.%, based on the total weight of the cosmetic composition. In further embodiments, the amount of water in the cosmetic composition is from about 55 to about 80 wt.%, about 55 to about 75 wt.%, about 60 to about 85 wt.%, about 60 to about 80 wt.%, about 60 to about 75 wt.%, about 65 to about 85 wt.%, about 65 to about 80 wt.%, about 65 to about 75 wt.%, based on the total weight of the cosmetic composition.

### (g) Water-Soluble Solvents

In various embodiments, the cosmetic compositions of the instant disclosure optionally include one or more water-soluble solvents. The term “water-soluble solvent” is interchangeable with the term “water-miscible solvent” and means a compound that is liquid at 25° C. and at atmospheric pressure (760 mmHg), and it has a solubility of at least 50% in water under these conditions. In an embodiment, the water-soluble solvents have a solubility of at least 60%, 70%, 80%, or 90%. Nonlimiting examples of water-soluble solvents include glycerin, mono-alcohols, polyols (polyhydric alcohols), glycols, and a mixture thereof. In an embodiment, the one or more water-soluble solvents may be chosen from glycerin, propylene glycol, butylene glycol, pentylene glycol, dipropylene glycol, ethanol, isopropanol, t-butyl alcohol, and a mixture thereof.

As nonlimiting examples of organic solvents, mention can be made of monoalcohols and polyols such as ethyl alcohol, isopropyl alcohol, propyl alcohol, isopropyl alcohol, benzyl alcohol, 4-tert-butylcyclohexanol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are also useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

In an embodiment, the cosmetic compositions include one or more glycols and/or one or more alcohols, for example, one or more water-soluble solvents selected from propylene glycol, butylene glycol, pentylene glycol, dipropylene glycol, ethanol, isopropanol, tert-butyl alcohol, and mixtures thereof.

The amount of the one or more water-soluble solvents, if present, will vary. Nonetheless, in various embodiments, the total amount of the one or more water-soluble solvents, if present, in the cosmetic compositions is from about 1 wt.% to about 20 wt.%, based on the total weight of the cosmetic composition. In further embodiments, the amount of the one or more water-soluble solvents in the cosmetic compositions is from about 1 to 18 wt.%, about 1 to about 15 wt.%, about 5 to about 20 wt.%, about 5 to about 20 wt.%, about 5 to about 15 wt.%, about 6 to about 20 wt.%, about 6 to about 18 wt.%, about 6 to about 15 wt.%, about 8 to about 20 wt.%, about 8 to about 18 wt.%, or about 8 to about 15 wt.%, based on the total weight of the cosmetic composition.

### (h) Miscellaneous Ingredients

The cosmetic compositions of the instant disclosure may optionally include one or more miscellaneous ingredients. Miscellaneous ingredients are ingredients that are compatible with the cosmetic compositions and do not disrupt or materially affect the basic and novel properties of the cosmetic compositions. Miscellaneous ingredients commonly used in cosmetics are known in the art. Non-limiting examples include miscellaneous emulsifiers/surfactants other than the nonionic emulsifiers of plurality of nonionic emulsifiers of (b), preservatives, fragrances, pH adjusters, salts, buffers, antioxidants, flavonoids, vitamins, botanical extracts, UV filtering agents, proteins, protein hydrolysates and/or isolates, hydrotropes, pearlescent agents, fillers, colorants, mattifying agents, further skin active agents, depigmenting agents, anti-wrinkle agents, etc. In a preferred embodiment, the cosmetic compositions of the instant disclosure include one or more skin active agents, in particular, madecassoside. Nonlimiting examples of various miscellaneous ingredients that may optionally be include (or excluded) from the cosmetic compositions is provided below.

### Miscellaneous Emulsifiers/Surfactants

Miscellaneous emulsifiers/surfactants may optionally be included in the cosmetic compositions. Miscellaneous emulsifiers/surfactants are those that other than the plurality of nonionic emulsifiers of (b). The miscellaneous emulsifiers/surfactants may be anionic, cationic, and/oramphoteric/zwitterionic.

### Antioxidants

Examples of antioxidants include tocopherols (e.g. d-α-tocopherol, d-β-tocopherol, d-γ-tocopherol, d-delta-tocopherol), tocotrienols (e.g. d-α-tocotrienol, d-β-tocotrienol, d-γ.-tocotrienol, d-delta-tocotrienol,) and vitamin E (α-tocopherol acetate). These compounds may be isolated from natural sources, prepared by synthetic means, or mixtures thereof. Tocotrienol-enriched vitamin E preparations may be obtained by fractionating vitamin E preparations to remove a portion of tocopherols and recover a preparation more highly concentrated in tocotrienol. Useful tocotrienols are natural products isolated, for example, from wheat germ oil, grain, or palm oil using high performance liquid chromatography, or isolated by alcohol extraction and/or molecular distillation from barley, brewer’s grain or oats. As used herein, the term “tocotrienols” includes tocotrienol-rich-fractions obtained from these natural products as well as the pure compounds. The increased glutathione peroxidase activity protects the skin from oxidative damage.

Vitamin C and derivatives may be used, including ascorbic acid, sodium ascorbate, and the fat-soluble esters tetrahexyldecyl ascorbate and ascorbyl palmitate, magnesium ascorbyl phosphate, ascorbyl-glucoside, glucosamine ascorbate, ascorbyl acetate, etc. Additionally, extracts from plants containing a high amount of vitamin C such as camu berry (Myrciaria dubia), acerola, emblica officinalis, and bioflavonoids from rose hip and citrus may be used including water soluble bioflavonoids such as hesperidin methyl chalcone may also be used.

Sesame (Sesamum indicum) or sesame lignan may also be added. Sesame and its lignans (the fibrous compounds associated with the sesame) act as antioxidants. Sesame seed lignans significantly enhance vitamin E activity.

In addition, carotenoids, particularly the xanthophyll type, are also useful antioxidants that can be used. The xanthopyll type carotenoids include molecules, such as lutein, canthaxantin, cryptoxanthin, zeaxanthin and astaxanthin. Xanthophylls protect compounds, such as vitamin A, vitamin E, and other carotenoids.

Flavonoids can also function as antioxidants. In some instances, the flavonoid is a flavanone (derivative of 2,3-dihydro-2-phenylchromen-4-one). Flavones include: Butin, Eriodictyol, Hesperetin, Hesperidin, Homoeriodictyol, Isosakuranetin, Naringenin, Naringin, Pinocembrin, Poncirin, Sakuranetin, Sakuranin, and Sterubin. The flavonoid may be a flavanonol (derivative of 3-hydroxy-2,3-dihydro-2-phenylchromen-4-one). Flavanols include: Taxifolin, Aromadedrin, Chrysandroside A, Chrysandroside B, Xeractinol, Astilbin, and Fustin. The flavonoid may be a flavone (derivative of 2-phenylchromen-4-one). Flavones include: Apigenin, Luteolin, Tangeritin, Chrysin, Baicalein, Scutellarein, Wogonin, Synthetic Flavones: Diosmin, and Flavoxate. The flavonoid may be a flavonol (derivative of 3-hydroxy-2-phenylchromen-4-one). Flavonols include: 3-Hydroxyflavone, Azaleatin, Fisetin, Galangin, Gossypetin, Kaempferide, Kaempferol, Isorhamnetin, Morin, Myricetin, Natsudaidain, Pachypodol, Quercetin, Rhamnazin, Rhamnetin, Azalein, Hyperoside, Isoquercitin, Kaempferitrin, Myricitrin, Quercitrin, Robinin, Rutin, Spiraeoside, Xanthorhamnin, Amurensin, Icariin, and Troxerutin. The flavonoid may be a flavan-3-ol (derivatives of 2-phenyl-3,4-dihydro-2H-chromen-3-ol). Flavan-3-ols include: Catechin, Epicatechin, Epigallocatechin, Epicatechin gallate, Epigallocatechin gallate, Epiafzelechin, Fisetinidol, Guibourtinidol, Mesquitol, and Robinetinidol. The flavonoid may be a flavan-4-ol (derivative of 2-phenylchroman-4-ol). Flavan-4-ols include: Apiforol and Luteoforol. The flavonoid may be an isoflavone (derivative of 3-phenylchromen-4-one). Isoflavones include: Genistein, Daidzein, Biochanin A, Formononetin, and the Equol metabolite from Daidzein.

The antioxidant may be an anthocyanidin (derivative of 2-phenylchromenylium cation). Anthocyanidins include: Aurantinidin, Cyanidin, Delphinidin, Europinidin, Luteolinidin, Pelargonidin, Malvidin, Peonidin, Petunidin, Rosinidin, and Xanthone.

The antioxidant may be a Dihydrochalcone (derivative of 1,3-diphenyl-1-propanone). Dihydrochalcones include: Phloretin, Dihydrochalcone phloretin Phlorizin, Aspalathin, Naringin dihydrochalcone, Neohesperidin dihydrochalcone, and Nothofagin. Without limiting the mode of action of the invention, dihydrochalcones may exert an antioxidant effect by reducing reactive free radicals, like reactive oxygen and reactive nitrogen species.

The antioxidant may be an anthocyanin. Anthocyanins and their derivatives are antioxidants. Anthocyanins encompasses a class of flavonoid compounds that are naturally occurring, water-soluble compounds, responsible for the red, purple, and blue colors of many fruits, vegetables, cereal grains, and flowers. Additionally, anthocyanins are collagenase inhibitors. The inhibition of collagenase helps in the prevention and reduction of wrinkles, increase in skin elasticity, etc., which are caused by a reduction in skin collagen. The anthocyanins may be obtained from any portion of various plant sources, such as the fruit, flower, stem, leaves, root, bark, or seeds. One of skill in the art will understand that certain portions of the plant may contain higher natural levels of anthocyanins, and, therefore, those portions are used to obtain the desired anthocyanins. In some instances, antioxidants may include one or more betacyanin. Betacyanins, like anthocyanins, may be obtained from natural sources and are antioxidants.

The antioxidant may be a Phenylpropanoid (derivatives of cinnamic acid). Phenylpropanoids include: Cinnamic acid, Caffeic acid, Ferulic acid, Trans-ferulic acid (including its antioxidant pharmacore 2,6-dihydroxyacetophenome), 5-Hydroxyferulic acid, Sinapic acid, Coumaryl alcohol, Coniferyl alcohol, Sinapyl alcohol, Eugenol, Chavicol, Safrole, P-coumaric acid, and Sinapinic acid. Without limiting the mode of action of the invention, Phenylpropanoids may neutralize free radicals.

The antioxidant may be a Chalcone (derivative of 1,3-diphenyl-2-propen-1-one). Chalcones include: Butein, Okanin, Carthamin, Marein, Sophoradin, Xanthohumol, Flavokvain A, Flavokavain B, Flavokavin C, and synthetic Safalcone.

The antioxidant may be a Curcuminoid. Curcuminoids include: Curcumin, Desmethoxycurcumin, bis-Desmethoxycurcumin, Tetrahydrocurcumin, and Tetrahydrocurcuminoids. Curcumin and tetrahydrocurcuminoids may be derived from rhizomes of Curcuma longa. Tetrahydrocurcumin, a metabolite of curcumin, has been found to be a more potent antioxidant and more stable compared to curcumin.

The antioxidant may be a Tannin. Tannins include: Tannin, Terflavin B, Glucogallin, Dgallic acid, and Quercitannic acid.

The antioxidant may be a stilbenoid. Stilbenoids include: Resveratrol, Pterostilbene, and Piceatannol. Resveratrol may include, but is not limited to, 3,5,4′-trihydroxystilbene, 3,4,3′,5′-tetrahydroxystilbene (piceatannol), 2,3′,4,5′-tetrahydroxystilbene (oxyresveratrol), 4,4′-dihydroxystilbene, and alpha and beta glucoside, galactoside and mannoside derivatives thereof.

The antioxidant may be a Coumarin (derivatives of 2H-chromen-2-one). Coumarins include: 4-Hydroxycoumarin, Umbelliferone, Aesculetin, Herniarin, Auraptene, and Dicoumarol.

The antioxidant may be a Carotenoid. Carotenoids include: beta-Carotene, alpha-Carotene, gamma-Carotene, beta-Cryptoxanthin, Lycopene, Lutein, and Idebenone. Sesame (Sesamum indicum) or sesame lignan may also be added. Sesame and its lignans (the fibrous compounds associated with the sesame) act as antioxidants. Sesame seed lignans significantly enhance vitamin E activity.

The antioxidant may be: a Xanthone, Butylated Hydroxytoluene, 2,6-Di-tert-butylphenol, 2,4-Dimethyl-6-tert-butylphenol, Gallic acid, Eugenol, Uric acid, alpha-Lipoic acid, Ellagic acid, Chicoric acid, Chlorogenic acid, Rosmarinic acid, Salicylic acid, Acetylcysteine, S-Allyl cysteine, Barbigerone, Chebulagic acid, Edaravone, Ethoxyquin, Glutathione, Hydroxytyrosol, Idebenone, Melatonin, N-Acetylserotonin, Nordihydroguaiaretic acid, Oleocanthal, Oleuropein, Paradol, Piceatannol, Probucol, Propyl gallate, Protocatechuic acid, Pyritinol, Rutin, Secoisolariciresinol diglucoside, Sesamin, Sesamol, Silibinin, Silymarin, Theaflavin, Theaflavin digallate, Thmoquinone, Trolox, Tyrosol, Polyunsaturated fatty acids, and sulfur-based antioxidants such as Methionine or Lipoic acid.

### Skin Active Agents

Nonlimiting examples of skin active agents include madecassoside, retinoic acid, benzoyl peroxide, sulfur, vitamin B6 (pyridoxine or) chloride, selenium, samphire--the cinnamon extract blends, tea and octanoylglycine such as--15 Sepicontrol A5 TEA from Seppic--the mixture of cinnamon, sarcosine and octanoylglycine marketed especially by Seppic under the trade name Sepicontrol A5--zinc salts such as zinc gluconate, zinc pyrrolidonecarboxylate (or zinc pidolate), zinc lactate, zinc aspartate, zinc carboxylate, zinc salicylate 20, zinc cysteate;--derivatives particularly copper and copper pidolate as Cuivridone Solabia--extracts from plants of Arnica montana, Cinchona succirubra, Eugenia caryophyllata, Humulus lupulus, Hypericum perforatum, Mentha pipenta 25 Rosmarinus officinalis, Salvia officinalis and Thymus vulgaris, all marketed for example by Maruzen--extracts of meadowsweet (Spiraea ulmaria), such as that sold under the name Sebonormine by Silab--extracts of the alga Laminaria saccharina, such as that sold under the 30 name Phlorogine by Biotechmarine--the root extracts of burnet mixtures (Sanguisorba officinalis/Poterium officinale), rhizomes of ginger (Zingiber officinalis) and cinnamon bark (Cinnamomum cassia), such as that sold under the name Sebustop by Solabia--extracts of flaxseed such as that sold under the name Linumine by Lucas Meyer--Phellodendron extracts such as those sold under the name Phellodendron extract BG by Maruzen or Oubaku liquid B by Ichimaru Pharcos--of argan oil mixtures extract of Serenoa serrulata (saw palmetto) extract and sesame seeds such as that sold under the name Regu SEB by Pentapharm--mixtures of extracts of willowherb, of Terminalia chebula, nasturtium and of bioavailable zinc (microalgae), such as that sold under the name Seborilys Green Tech;--extracts of Pygeum afrianum such as that sold under the name Pygeum afrianum sterolic lipid extract by Euromed--extracts of Serenoa serrulata such as those sold under the name Viapure Sabal by Actives International, and those sold by the company Euromed--of extracts of plantain blends, Berberis aquifolium and sodium salicylate 20 such as that sold under the name Seboclear Rahn--extract of clove as that sold under the name Clove extract powder by Maruzen--argan oil such as that sold under the name Lipofructyl Laboratories Serobiologiques; 25--lactic protein filtrates, such as that sold under the name Normaseb by Sederma--the seaweed laminaria extracts, such as that sold under the name Laminarghane by Biotechmarineoligosaccharides seaweed Laminaria digitata, such as that sold under the name Phycosaccharide 30 AC by the company Codif--extracts of sugar cane such as that sold under the name Policosanol by the company Sabinsa, the sulfonated shale oil, such as that sold under the name Ichtyol Pale by Ichthyol--extracts of meadowsweet (Spiraea ulmaria) such as that sold under the name Cytobiol Ulmaire by societeLibiol--sebacic acid, especially sold in the form of a sodium polyacrylate gel under the name Sebosoft by Sederma--glucomannans extracted from konjac tuber and modified with alkylsulfonate chains such as that sold under the name Biopol Beta by Arch Chemical--extracts of Sophora angustifolia, such as those sold under the name Sophora powder or Sophora extract by Bioland--extracts of cinchona bark succirubra such as that sold under the name Red Bark HS by Alban Muller--extracts of Quillaja saponaria such as that sold under the name 15 Panama wood HS by Alban Muller--glycine grafted onto an undecylenic chain, such as that sold under the name Lipacide UG OR by SEPPIC--the mixture of oleanolic acid and nordihydroguaiaretic acid, such as that sold under the form of a gel under the name AC.Net by Sederma; 20--phthalimidoperoxyhexanoic acidcitrate tri (C12-C13) sold under the name COSMACOL.RTM ECI by Sasol; trialkyl citrate (C14-C15) sold under the name COSMACOL.RTM. ECL by Sasol--10-hydroxydecanoic acid, including mixtures acid-hydroxydecanoic October 25, sebacic acid and 1,10-decandiol such as that sold under the name Acnacidol BG by Vincience and mixtures thereof.

### Depigmenting Agents

Nonlimiting examples of depigmenting agents include alpha and beta arbutin, ferulic acid, lucinol and its derivatives, kojic acid, resorcinol and derivatives thereof, tranexamic acid and derivatives thereof, gentisic acid, homogentisic, methyl gentisate or homogentisate, dioic acid, D pantheteine calcium sulphonate, lipoic acid, ellagic acid, vitamin B3, linoleic acid and its derivatives, certain compounds derived from plants such as chamomile, bearberry, the aloe family (vera, ferox, bardensis), mulberry, skullcap, a water kiwi fruit (Actinidia chinensis) marketed by Gattefosse, an extract of Paeonia suffruticosa root, such as that sold by Ichimaru Pharcos under the name Liquid Botanpi Be an extract of brown sugar (Saccharum officinarum) such as molasses extract marketed by Taiyo Kagaku under the name Liquid Molasses, without this list being exhaustive. Particular depigmenting agents include alpha and beta arbutin, ferulic acid, kojic acid, resorcinol and derivatives, D pantheteine calcium sulfonate, lipoic acid, ellagic acid, vitamin B3, a water kiwi fruit (Actinidia chinensis) marketed by Gattefosse, an extract of Paeonia suffruticosa root, such as that sold by the company Ichimaru Pharcos under the name Botanpi Liquid B.

### Anti-Wrinkle Agent

The term “anti-wrinkle agent” refers to a natural or synthetic compound producing a biological effect, such as the increased synthesis and/or activity of certain enzymes, when brought into contact with an area of wrinkled skin, this has the effect of reducing the appearance of wrinkles and/or fine lines. Nonlimiting examples of anti-wrinkle agents include: desquamating agents, anti-glycation agents, inhibitors of NO-synthase, agents stimulating the synthesis of dermal or epidermal macromolecules and/or preventing their degradation, agents for stimulating the proliferation of fibroblasts and/or keratinocytes, or for stimulating keratinocyte differentiation reducing agents; muscle relaxants and/or dermo-decontracting agents, anti-free radical agents, and mixtures thereof. Examples of such compounds are: adenosine and its derivatives and retinoids other than retinol (as discussed above, such as retinol palmitate), ascorbic acid and its derivatives such as magnesium ascorbyl phosphate and ascorbyl glucoside; nicotinic acid and its precursors such as nicotinamide; ubiquinone; glutathione and precursors thereof such as L-2-oxothiazolidine-4-carboxylic acid, the compounds C-glycosides and their derivatives as described in particular in EP-1345919, in particular C-beta-D-xylopyranoside-2-hydroxy-propane as described in particular in EP-1345919, plant extracts including sea fennel and extracts of olive leaves, as well as plant and hydrolysates thereof such as rice protein hydrolysates or soybean proteins; algal extracts and in particular laminaria, bacterial extracts, the sapogenins such as diosgenin and extracts of Dioscorea plants, in particular wild yam, comprising: the a-hydroxy acids, f3-hydroxy acids, such as salicylic acid and n-octanoyl-5-salicylic oligopeptides and pseudodipeptides and acyl derivatives thereof, in particular acid {2-[acetyl-(3-trifluoromethyl-phenyl)-amino]-3-methyl-}acetic acid and lipopeptides marketed by the company under the trade names SEDERMA Matrixyl 500 and Matrixyl 3000; lycopene, manganese salts and magnesium salts, especially gluconates, and mixtures thereof. In at least one case, the skin tightening composition includes adenosine derivatives, such as non-phosphate derivatives of adenosine, such as in particular the 2′-deoxyadenosine, 2′,3′-adenosine isopropoylidene; the toyocamycine, 1-methyladenosine, N-6-methyladenosine; adenosine N-oxide, 6-methylmercaptopurine riboside, and the 6-chloropurine riboside. Other derivatives include adenosine receptor agonists such as adenosine phenylisopropyl (“PIA”), 1-methylisoguanosine, N6-cyclohexyladenosine (CHA), N6-cyclopentyladenosine (CPA), 2-chloro-N6-cyclopentyladenosine, 2-chloroadenosine, N6-phenyladenosine, 2-phenylaminoadenosine, MECA, N 6-phenethyladenosine, 2-p-(2-carboxy-ethyl) phenethyl-amino-5′- -N-ethylcarboxamido adenosine (CGS-21680), N-ethylcarboxamido-adenosine (NECA), the 5′(N-cyclopropyl)-carboxamidoadenosine, DPMA (PD 129.944) and metrifudil.

Miscellaneous ingredients can be included in the cosmetic composition, for example, in an amount of about 0.01 to about 10 wt.%, based on the total weight of the cosmetic composition. The total amount of the one or more miscellaneous ingredients may be about 0.01 to about 8 wt.%, about 0.01 to about 5 wt.%, about 0.01 to about 3 wt.%, about 0.1 to about 10 wt.%, about 0.1 to about 8 wt.%, about 0.1 to about 5 wt.%, about 0.1 to about 3 wt.%, about 1 to about 10 wt.%, about 1 to about 8 wt.%, about 1 to about 5 wt.%, or about 1 to about 3 wt.%, based on the total weight of the cosmetic composition.

As already noted, skin active agents may be included as one or more of the miscellaneous ingredients. With respect to the total amount of skin active agents in the cosmetic compositions, if present, the total amount of skin active agents may be from greater than zero to about 9 wt.%, greater than zero to about 8 wt.%, greater than zero to about 7 wt.%, greater than zero to about 6 wt.%, greater than zero to about 5 wt.%, greater than zero to about 4 wt.%, greater than zero to about 3 wt.%, greater than zero to about 2 wt.%; about 10 ppm to about 10 wt.% (100,000 ppm), about 10 ppm to about 5 wt.% (50,000 ppm), about 10 ppm to about 2.5 wt.% (25,000 ppm), about 10 ppm to about 1 wt.% (10,000 ppm), about 10 ppm to about 0.5 wt.% (5,000 ppm), about 10 ppm to about 0.3 wt.% (3,000 ppm), about 10 ppm to about 0.2 wt.% (2,000 ppm), about 10 ppm to about 0.1 wt.% (1,000 ppm), about 10 ppm to 500 ppm; about 0.1 to about 10 wt.%, about 0.1 to about 5 wt.%, about 0.1 to about 2.5 wt.%, about 0.1 to about 1 wt.%, about 0.1 to about 0.5 wt.%; about 1 to about 10 wt.%, about 1 to about 8 wt.%, about 1 to about 6 wt.%, about 1 to about 5 wt.%, about 1 to about 4 wt.%, about 1 to about 3 wt.%; about 2 to about 10 wt.%, about 2 to about 8 wt.%, about 2 to about 6 wt.%, about 2 to about 5 wt.%, about 2 to about 4 wt.%; about 3 to about 10 wt.%, about 3 to about 8 wt.%, about 3 to about 6 wt.%, about 3 to about 5 wt.%; about 4 to about 10 wt.%, about 4 to about 8 wt.%, or about 4 to about 6 wt.%, based on the total weight of the cosmetic composition.

In an embodiment, the cosmetic compositions of the instant disclosure may optionally include one or more phenolic or polyphenolic active compounds, for example, baicalin. However, in an embodiment, the cosmetic compositions may be free or essentially free from phenolic or polyphenolic active compounds, in particular baicalin.

### Stability

The cosmetic compositions of the instant disclosure are stable, and retinol is preserved. With respect to physical stability, in certain embodiments, the cosmetic compositions do not visually phase separate or form visibly observable particulates for at least 2 weeks, 4 weeks, and/or 8 weeks in storage at 4° C., 25° C., 37° C., and/or 45° C.

In another embodiment, the cosmetic compositions do not visually phase separate or form visibly observable particulates for at least 10 cycles of freeze-thaw testing, wherein the freeze-thaw testing comprises placing the cosmetic composition in a stability chamber and subjecting it to temperature fluctuation at 12-hour intervals, for a first interval of 12 hours at -20° C. followed by a second interval of 12 hours at 25° C.

In another embodiment, the viscosity of the cosmetic compositions does not change by more than 20%, 15%, 10%, or 5%, for at least 2 weeks, 4 weeks, and/or 8 weeks in storage at 4° C., 25° C., 37° C., and/or 45° C.

With respect to chemical stability of retinol, in various embodiments, at least 70%, 75%, 80%, 85%, 90%, or 95% of retinol is preserved at 8 weeks when the cosmetic composition is incorporated into a sealed polyethylene terephthalate glycol (PETG) container and stored at 25° C. and/or 45° C.

### Viscosity

In general, it is preferably that the cosmetic compositions of the instant case have a viscosity of about 10,000 to about 200,000 Pa.s at 25° C., and shear rate of 1 s⁻¹ at 25° C. However, the cosmetic compositions may have a viscosity of about 10,000 to about 200,000 Pa.s, about 10,000 to about 180,000 Pa.s, about 10,000 to about 150,000 Pa.s, about 10,000 to about 120,000 Pa.s, about 15,000 to about 200,000 Pa.s, about 15,000 to about 180,000 Pa.s, about 15,000 to about 150,000 Pa.s, about 15,000 to about 120,000 Pa.s, about 20,000 to about 200,000 Pa.s, about 20,000 to about 180,000 Pa.s, about 20,000 to about 150,000 Pa.s, or about 20,000 to about 120,000 Pa.s at 25° C., and shear rate of 1 s⁻¹ at 25° C.

The viscosity measurements can be carried out, for example, using a Broooksfield viscometer/rheometer using a t-bar spindle at a speed of 5, 10, 15, and/or 20 rpm. An RVDV-II+Pro Viscometer with RheocalcT software may be employed for automated instrument control and data acquisition. The test temperature is maintained at 25° C. by using a Brookfield TC-502P Programmable Refrigerated Bath. From its original container, a sample is transferred into a 120 mL glass jar and then tested.

### Methods

The instant disclosure relates to methods of treating skin. The methods include applying a cosmetic composition according to the instant disclosure, optionally allowing the cosmetic composition to remain on the skin for a period of time. The cosmetic compositions are typically applied directly to the skin using the hand or a cloth. The skin may be optionally washed or rinsed prior to application. The method for treating the skin can be carried out once daily or may be carried out multiple times. For example, the method for treating skin may be carried out once daily, twice daily, weekly, bi-weekly for an extended period of time, for example, for about 1, 2, 3, 4, 5, or 6 months up to 1 year, or longer. In various embodiments, the methods are directed to, but are not limited to, boosting collagen and/or elastin production, reducing the appearance of fine lines, wrinkles, and uneven skin tone, tightening the skin, preventing/or treating acne, decreasing the development of melanin, and improving and brightening overall complexion.

In certain embodiments, the method further comprises mixing a cosmetic composition of the instant disclosure with one or more additional cosmetic compositions prior to application to the skin. For example, the cosmetic composition of the instant disclosure can be mixed with one or more additional cosmetic compositions immediately prior to application to the skin, for example, the mixing may occur within 1 minute, 2 minutes, 5 minutes, 10 minutes, 15 minutes, or 30 minutes prior to application to the skin. In certain embodiments, the cosmetic compositions can be mixed in an individual’s hands prior to applying the mixture to the skin, for example, the skin of the face.

In various embodiments, the instant disclosure relates to methods for stabilizing cosmetic composition containing high amounts of retinol. In various embodiments, the instant disclosure relates to preserving retinol in cosmetic compositions. This results in cosmetic compositions containing high amounts of retinol that preserve (maintain) the high amounts of retinol over time.

### Kits

The cosmetic compositions of the instant disclosure may be provided in a kit, for example, a kit comprising an individually contained cosmetic composition according to the instant disclosure and one or more additional separately contained cosmetic compositions. In an embodiment, the one or more separately contained compositions may be an additional composition according to the instant disclosure or may be a different composition. The cosmetic compositions may be separately contained in different cartridges, which are included in a dispensing apparatus/device. In other words, the kit may be a dispensing apparatus/device comprising a plurality of cartridges in which the compositions are contained. The kit (or apparatus/device) may optionally dispense the cosmetic composition of the instant disclosure and separately dispense the one or more separately contained composition. In various embodiments, the compositions may be dispensed individually or concurrently, and may optionally be mixed (or not mixed) with each other prior to being dispensed. In an embodiment, the various compositions are not mixed with each other prior to being dispensed. Useful systems, cartridges, and dispensing apparatus/devices are disclosed in U.S. Pat. Nos. 9,968,177 and 9,808,071; U.S. Pat. Application Publication. Nos. 2021/0236390, 2021/0235849 and 2021/0236863; and in U.S. Serial No. 17/162,555, which are all incorporated herein by reference in their entirety.

### Embodiments

In some embodiments, the cosmetic composition comprises or consists of:
(a) about 0.05 to about 6 wt.%, preferably from about 0.1 to about 5 wt.%, more preferably about 0.5 to about 3 wt.% of retinol;
(b) about 1 to about 8 wt.%, preferably about 1.3 to about 8 wt.%, more preferably about 1.3 to about 6 wt.%, even more preferably about 1.3 to about 5 wt.% of a plurality of nonionic emulsifiers comprising:
   (i) one or more ethoxylated fatty acids, for example, adducts of ethylene oxide with esters of lauric acid, palmitic acid, stearic acid or behenic acid, and mixtures thereof, having from 9 to 100 oxyethylene groups;
   (ii) one or more nonionic emulsifiers having a Hydrophile-Lipophile Balance (HLB) from about 9 to about 12, for example, chosen from: (1) polyglyceryl fatty acid esters, polyoxyalkylenated alkyl glycerides, and polyoxyalkylenated fatty ethers; (2) mixed esters of fatty acid or of fatty alcohol, of carboxylic acid and of glycerol; (3) fatty acid esters of sugars and fatty alcohol ethers of sugars; (4) fatty esters of sorbitan and oxyalkylenated fatty esters of sorbitan; and mixtures thereof;
   (iii) optionally, one or more glyceryl esters, for example, glyceryl behenate, glyceryl erucate, glyceryl hydroxystearate, glyceryl isostearate, glyceryl lanolate, glyceryl laurate, glyceryl myristate, glyceryl palmitate lactate, glyceryl sesquioleate, glyceryl stearate, glyceryl distearate, glyceryl laurate, or a mixture thereof. In at least one instance the glyceryl ester comprises glyceryl stearate, glyceryl ricinoleate, and mixtures thereof;
   (iv) optionally, one or more additional nonionic emulsifiers;
(c) about 0.1 to about 8 wt.%, preferably about 0.5 to about 6 wt.%, more preferably about 1 to about 5 wt.% of one or more fatty alcohols, for example, fatty alcohols chosen from fatty alcohols having from 8 to 24 carbon atoms, in particular, C14-24 alcohols, cetyl alcohol, stearyl alcohol, cetearyl alcohol, behenyl alcohol, lauryl alcohol, myristic or myristyl
alcohol, arachidyl alcohol, lignoceryl alcohol, and mixtures thereof; (d) about 1 to about 20 wt.%, preferably about 5 to about 18 wt.%, more preferably about 5 to about 15 wt.% of one or more fatty compounds, for example, chosen from fatty esters (e.g., isononyl isononanoate), polyolefins (petrolatum), waxes, squalane, squalene, hydrogenated polyisobutene, hydrogenated polydecene, polybutene, mineral oil, pentahydrosqualene, plant and/or vegetable oil (e.g., soybean oil), hydrocarbon-based oils (e.g., isohexadecane), silicone oils, and a mixture thereof;
(e) about 0.1 to about 8 wt.%, preferably about 0.5 to about 5 wt.%, more preferably about 1 to about 3 wt.% of one or more thickening polymers, preferably one or more taurate copolymers, in particular, one or more taurate copolymers chosen from acrylamide/sodium acryloyl dimethyl taurate copolymer, hydroxyethyl acrylate/sodium acryloyl dimethyl taurate copolymer, ammonium acryloyldimethyl taurate/VP copolymer, sodium acrylate/sodium acryloyl dimethyl taurate copolymer, hydroxyethyl acrylate/ sodium acryloyldimethyl taurate copolymer, and a mixture thereof; and
(f) about 55 to about 85 wt.%, preferably about 60 to about 80 wt.%, more preferably about 65 to about 75 wt.% of water;
(g) optionally, one or more water-soluble solvents, for example, one or more water-soluble solvents chosen glycerin, mono-alcohols (for example, C1-5 mono-alcohols), organic solvents, polyols (polyhydric alcohols), glycols (e.g., propylene glycol, butylene glycol, pentylene glycol, etc.), and a mixture thereof, preferably glycerin, one or more monoalcohols chosen from ethanol, isopropanol, and t-butyl alcohol, and one or more glycols chosen from propylene glycol, butylene glycol, and pentylene glycol, wherein if present, the amount of the one or more water-soluble solvents is from about 1 to about 20 wt.%, preferably about 5 to about 18 wt.%, more preferably about 5 to about 15 wt.%;
(h) optionally, one or more miscellaneous ingredients, for example, one or more miscellaneous ingredients chosen from miscellaneous emulsifiers/surfactants other than the nonionic emulsifiers of plurality of nonionic emulsifiers of (b), preservatives, fragrances, pH adjusters, salts, buffers, antioxidants, flavonoids, vitamins, botanical extracts, UV filtering agents, proteins, protein hydrolysates and/or isolates, hydrotropes, pearlescent agents, fillers, colorants, mattifying agents, further skin active agents, depigmenting agents, anti-wrinkle agents, wherein if present, the one or more miscellaneous ingredients comprise about 0.01 to about 0.1 to about 10 wt.%, preferably about 0.1 to about 8 wt.%, more preferably about 1 to about 8 wt.% of the cosmetic composition;
   wherein the composition is in the form of an oil in water emulsion and all percentages by weight are based on the total weight of the cosmetic composition.

In certain preferred embodiments, the plurality of nonionic emulsifiers (b) may comprise or consist of:
(i) about 0.1 to about 5 wt.%, preferably about 0.1 to about 3 wt.%, more preferably about 0.2 to about 2 wt.% of one or more ethoxylated fatty acids, for example, adducts of ethylene oxide with esters of lauric acid, palmitic acid, stearic acid or behenic acid, and mixtures thereof, having from 9 to 100 oxyethylene groups;
(ii) about 0.1 to about 5 wt.%, preferably about 0.1 to about 3 wt.%, more preferably about 0.2 to about 2 wt.% of one or more nonionic emulsifiers having a Hydrophile-Lipophile Balance (HLB) from about 9 to about 12, for example, chosen from: (1) polyglyceryl fatty acid esters, polyoxyalkylenated alkyl glycerides, and polyoxyalkylenated fatty ethers; (2) mixed esters of fatty acid or of fatty alcohol, of carboxylic acid and of glycerol; (3) fatty acid esters of sugars and fatty alcohol ethers of sugars; (4) fatty esters of sorbitan and oxyalkylenated fatty esters of sorbitan; and mixtures thereof;
(iii) optionally, about 0.1 to about 5 wt.%, preferably about 0.1 to about 3 wt.%, more preferably about 0.2 to about 2 wt.% of one or more glyceryl esters, for example, glyceryl behenate, glyceryl erucate, glyceryl hydroxystearate, glyceryl isostearate, glyceryl laurate, glyceryl linoleate, glyceryl myristate, glyceryl palmitate lactate, glyceryl stearate, glyceryl distearate, glyceryl laurate, or a mixture thereof, preferably chosen from glyceryl stearate, glyceryl ricinoleate, and mixtures thereof;
(iv) optionally, one or more additional nonionic emulsifiers, wherein if present, the one or more additional nonionic emulsifiers may be in an amount of about 0.01 to about 5 wt.%, preferably about 0.01 to about 3 wt.%,

wherein all percentages by weight are based on the total weight of the cosmetic composition (not the total weight of the plurality of nonionic surfactants).

The cosmetic compositions of the instant disclosure are stable, and retinol is preserved. With respect to physical stability, in certain embodiments, the cosmetic compositions do not visually phase separate or form visibly observable particulates for at least 2 weeks, 4 weeks, and/or 8 weeks in storage at 4° C., 25° C., 37° C., and/or 45° C.

In another embodiment, the cosmetic compositions do not visually phase separate or form visibly observable particulates for at least 10 cycles of freeze-thaw testing, wherein the freeze-thaw testing comprises placing the cosmetic composition in a stability chamber and subjecting it to temperature fluctuation at 12-hour intervals, for a first interval of 12 hours at -20° C. followed by a second interval of 12 hours at 25° C.

In another embodiment, the viscosity of the cosmetic compositions does not change by more than 20%, 15%, 10%, or 5%, for at least 2 weeks, 4 weeks, and/or 8 weeks in storage at 4° C., 25° C., 37° C., and/or 45° C.

With respect to chemical stability of retinol, in various embodiments, at least 70%, 75%, 80%, 85%, 90%, or 95% of retinol is preserved at 8 weeks when the cosmetic composition is incorporated into a sealed polyethylene terephthalate glycol (PETG) container and stored at 25° C. and/or 45° C.

In general, the cosmetic compositions preferably have a viscosity of about 10,000 to about 200,000 Pa.s at 25° C., and shear rate of 1 s⁻¹ at 25° C. However, the cosmetic compositions may have a viscosity of about 10,000 to about 200,000 Pa.s, about 10,000 to about 180,000 Pa.s, about 10,000 to about 150,000 Pa.s, about 10,000 to about 120,000 Pa.s, about 15,000 to about 200,000 Pa.s, about 15,000 to about 180,000 Pa.s, about 15,000 to about 150,000 Pa.s, about 15,000 to about 120,000 Pa.s, about 20,000 to about 200,000 Pa.s, about 20,000 to about 180,000 Pa.s, about 20,000 to about 150,000 Pa.s, or about 20,000 to about 120,000 Pa.s at 25° C., and shear rate of 1 s⁻¹ at 25° C.

In various embodiments, the cosmetic composition comprises or consists of:
(a) about 0.05 to about 6 wt.%, preferably from about 0.1 to about 5 wt.%, more preferably about 0.5 to about 3 wt.% of retinol;
(b) about 1 to about 8 wt.%, preferably about 1.3 to about 8 wt.%, more preferably about 1.3 to about 6 wt.%, even more preferably about 1.3 to about 5 wt.% of a plurality of nonionic emulsifiers comprising:
   (ii) about 0.1 to about 5 wt.%, preferably about 0.1 to about 3 wt.%, more preferably about 0.2 to about 2 wt.% of one or more ethoxylated fatty acids, for example, adducts of ethylene oxide with esters of lauric acid, palmitic acid, stearic acid or behenic acid, and mixtures thereof, having from 9 to 100 oxyethylene groups;
   (ii) about 0.1 to about 5 wt.%, preferably about 0.1 to about 3 wt.%, more preferably about 0.2 to about 2 wt.% of one or more nonionic emulsifiers having a Hydrophile-Lipophile Balance (HLB) from about 9 to about 12, for example, chosen from: (1) polyglyceryl fatty acid esters, polyoxyalkylenated alkyl glycerides, and polyoxyalkylenated fatty ethers; (2) mixed esters of fatty acid or of fatty alcohol, of carboxylic acid and of glycerol; (3) fatty acid esters of sugars and fatty alcohol ethers of sugars; (4) fatty esters of sorbitan and oxyalkylenated fatty esters of sorbitan; and mixtures thereof, preferably at least one of the one or more nonionic emulsifiers having a Hydrophile-Lipophile Balance (HLB) from about 9 to about 12 is chosen from alkylpolyglucosides (e.g., C12-20 alkyl glucoside), polyglyceryl-10 laurate, polyglyceryl-2 dipolyhydroxystearate, polyglyceryl-2 polyhydroxystearate, polyglyceryl-3 laurate, polyglyceryl-3 methylglucose distearate, polyglyceryl-3 oleate, polyglyceryl-3 palmitate, polyglyceryl-3 polyricinoleate, polyglyceryl-3 ricinoleate, and mixture sthereof;
   (iii) optionally, about 0.1 to about 5 wt.%, preferably about 0.1 to about 3 wt.%, more preferably about 0.2 to about 2 wt.% of one or more glyceryl esters, for example, glyceryl behenate, glyceryl erucate, glyceryl hydroxystearate, glyceryl isostearate, glyceryl laurate, glyceryl myristate, glyceryl palmitate lactate, glyceryl stearate, glyceryl distearate, glyceryl laurate, or a mixture thereof, preferably chosen from glyceryl stearate, glyceryl ricinoleate, and mixtures thereof;
   (iv) optionally, one or more additional nonionic emulsifiers, wherein if present, the one or more additional nonionic emulsifiers may be in an amount of about 0.01 to about 5 wt.%, preferably about 0.01 to about 3 wt.%;
(c) about 0.1 to about 8 wt.%, preferably about 0.5 to about 6 wt.%, more preferably about 1 to about 5 wt.% of one or more fatty alcohols, for example, fatty alcohols chosen from fatty alcohols having from 8 to 24 carbon atoms, in particular, C14-24 alcohols, cetyl alcohol, stearyl alcohol, cetearyl alcohol, behenyl alcohol, lauryl alcohol, myristic or myristyl alcohol, arachidyl alcohol, lignoceryl alcohol, and mixtures thereof;
(d) about 1 to about 20 wt.%, preferably about 5 to about 18 wt.%, more preferably about 5 to about 15 wt.% of one or more fatty compounds, for example, chosen from fatty esters (e.g., isononyl isononanoate), polyolefins (petrolatum), waxes, squalane, squalene, hydrogenated polyisobutene, hydrogenated polydecene, polybutene, mineral oil, pentahydrosqualene, plant and/or vegetable oil (e.g., soybean oil), hydrocarbon-based oils (e.g., isohexadecane), silicone oils, and a mixture thereof;
(e) about 0.1 to about 8 wt.%, preferably about 0.5 to about 5 wt.%, more preferably about 1 to about 3 wt.% of one or more thickening polymers, wherein at least one of the one or more thickening polymers is a taurate copolymers chosen from acrylamide/sodium acryloyl dimethyl taurate copolymer, hydroxyethyl acrylate/sodium acryloyl dimethyl taurate copolymer, ammonium acryloyldimethyl taurate/VP copolymer, sodium acrylate/sodium acryloyl dimethyl taurate copolymer, hydroxyethyl acrylate/ sodium acryloyldimethyl taurate copolymer, and a mixture thereof; and
(f) about 55 to about 85 wt.%, preferably about 60 to about 80 wt.%, more preferably about 65 to about 75 wt.% of water;
(e) optionally, one or more water-soluble solvents, for example, one or more water-soluble solvents chosen glycerin, mono-alcohols (for example, C1-5 mono-alcohols), organic solvents, polyols (polyhydric alcohols), glycols (e.g., propylene glycol, butylene glycol, pentylene glycol, etc.), and a mixture thereof, preferably glyceryn, one or more monoalcohols chosen from ethanol, isopropanol, and t-butyl alcohol, and one or more glycols chosen from propylene glycol, butylene glycol, and pentylene glycol, wherein if present, the amount of the one or more water-soluble solvents is preferably about 1 to about 20 wt.%, preferably about 5 to about 18 wt.%, more preferably about 5 to about 15 wt.%;
(h) optionally, one or more miscellaneous ingredients, for example, one or more miscellaneous ingredients chosen from miscellaneous emulsifiers/surfactants other than the nonionic emulsifiers of plurality of nonionic emulsifiers of (b), preservatives, fragrances, pH adjusters, salts, buffers, antioxidants, flavonoids, vitamins, botanical extracts, UV filtering agents, proteins, protein hydrolysates and/or isolates, hydrotropes, pearlescent agents, fillers, colorants, mattifying agents, further skin active agents, depigmenting agents, anti-wrinkle agents, wherein if present, the one or more miscellaneous ingredients comprise about 0.01 to about 0.1 to about 10 wt.%, preferably about 0.1 to about 8 wt.%, more preferably about 1 to about 8 wt.% of the cosmetic composition;
   wherein the composition is in the form of an oil in water emulsion and all percentages by weight are based on the total weight of the cosmetic composition.

The cosmetic compositions of the instant disclosure are stable, and retinol is preserved. With respect to physical stability, in certain embodiments, the cosmetic compositions do not visually phase separate or form visibly observable particulates for at least 2 weeks, 4 weeks, and/or 8 weeks in storage at 4° C., 25° C., 37° C., and/or 45° C.

In another embodiment, the cosmetic compositions do not visually phase separate or form visibly observable particulates for at least 10 cycles of freeze-thaw testing, wherein the freeze-thaw testing comprises placing the cosmetic composition in a stability chamber and subjecting it to temperature fluctuation at 12-hour intervals, for a first interval of 12 hours at -20° C. followed by a second interval of 12 hours at 25° C.

In another embodiment, the viscosity of the cosmetic compositions does not change by more than 20%, 15%, 10%, or 5%, for at least 2 weeks, 4 weeks, and/or 8 weeks in storage at 4° C., 25° C., 37° C., and/or 45° C.

With respect to chemical stability of retinol, in various embodiments, at least 70%, 75%, 80%, 85%, 90%, or 95% of retinol is preserved at 8 weeks when the cosmetic composition is incorporated into a sealed polyethylene terephthalate glycol (PETG) container and stored at 25° C. and/or 45° C.

In general, the cosmetic compositions preferably have a viscosity of about 10,000 to about 200,000 Pa.s at 25° C., and shear rate of 1 s⁻¹ at 25° C. However, the cosmetic compositions may have a viscosity of about 10,000 to about 200,000 Pa.s, about 10,000 to about 180,000 Pa.s, about 10,000 to about 150,000 Pa.s, about 10,000 to about 120,000 Pa.s, about 15,000 to about 200,000 Pa.s, about 15,000 to about 180,000 Pa.s, about 15,000 to about 150,000 Pa.s, about 15,000 to about 120,000 Pa.s, about 20,000 to about 200,000 Pa.s, about 20,000 to about 180,000 Pa.s, about 20,000 to about 150,000 Pa.s, or about 20,000 to about 120,000 Pa.s at 25° C., and shear rate of 1 s⁻¹ at 25° C.

### EXAMPLES

As various changes could be made in the above-described compositions and methods without departing from the scope of the invention, it is intended that all matter contained in the above description and in the examples given below, shall be interpreted as illustrative and not in a limiting sense.

The following Examples are intended to be non-restrictive and explanatory only. The ingredient amounts in the compositions/formulas described below are expressed in % by weight, based on the total weight of the composition.

### Example 1

Inventive Compositions

| | | | | Inventive | |
|---|---|---|---|---|---|
| | | | | A | B |
| (a) | | Active | RETINOL | 0.6 | 0.6 |
| (b) | (i) | Ethoxylated Fattv Acid | PEG-40 STEARATE | 0.4 | 0.4 |
| | (ii) | Nonionic Emulsifier (HLB 9-12) | POLYGLYCERYL-3 METHYLGLUCOSE DISTEARATE | 0.5 | 0.5 |
| | | | C12-20 ALKYL GLUCOSIDE | 0.2 | 0.2 |
| | (iii) | Glyceryl Ester | GLYCERYL STEARATE | 0.3 | 0.3 |
| | (iv) | Nonionic Emulsifier | POLYSORBATE 60 | 0.04 | 0.04 |
| (c) | | Fatty Alcohol | CETYL ALCOHOL, AND C14-22 ALCOHOLS | 1.1 | 1.1 |
| (d) | | Fatty Compounds | GLYCINE SOJA (SOYBEAN) OIL, | 5.4 | 5.4 |
| | | | ISONONYL ISONONANOATE, | 3 | 3 |
| | | | ISOHEXADECANE, AND/OR DIISOPROPYL SEBACATE | 3 | 3 |
| | | Silicone Oil | DIMETHICONOL AND/OR DIMETHICONE | 1 | 0.5 |
| (e) | | Thickening Polymers | HYDROXYETHYL ACRYLATE/SODIUM ACRYLOYLDIMETHYL TAURATE COPOLYMER AND/OR AMMONIUM ACRYLOYLDIMETHYLTAURA TE/VP COPOLYMER | 1.4 | 1.4 |
| (g) | | Water-Soluble Solvent | T-BUTYL ALCOHOL, GLYCERIN, AND/OR PROPYLENE GLYCOL | 12 | 12 |
| (h) | | Miscellaneous emulsifers/surfactants, salts, preservatives, pH adjusters, fragrances, colorants, antioxidants, chelants, and/or extracts, etc. | | ≤ 4 | ≤ 4 |
| (f) | | WATER | | ~ 70 | ~ 70 |
| Physically Stable | | | | Y | Y |
| Chemically Stable (retinol) | | | | Y | Y |

### Example 2

### Stability Data

The compositions of Example 1 were subjected to physical stability studies and visually evaluated for phase separation and assessed under a microscope for particulate formation. The compositions were analyzed upon initial manufacture of the composition (T₀). The compositions were again analyzed after 10 days of freeze-thaw testing. For freeze-thaw testing, the compositions were placed in a stability chamber and subjected to temperature fluctuation at 12-hour intervals. For 12 hours, the compositions were held at -20° C. For the next 12 hours, the compositions were held at 25° C. The cycle was repeated 10 times (for 10 days). Separately, the compositions of Example 1 were evaluated after 4 weeks (1 month) in storage at 4° C., 25° C., 37° C., and 45° C. and again at 8 weeks (2 months) at 4° C., 25° C., 37° C., and 45° C. and visually evaluated for phase separation and assessed under a microscope for particulate formation.

The inventive compositions were deemed stable (“Y”) (yes) because they did not visually phase separate and did not form particulates.

Chemical stability was also evaluated, in particular, the chemical stability of retinol in the cosmetic composition (resistance to degradation). The compositions of Example 1 were individually added into sealed polyethylene terephthalate glycol (PETG) cartridges and stored at 25° C. and 45° C. and the amount of remaining retinol determined at 8 weeks. The amount of retinol was determined by HPLC coupled with UV-Vis. A calibration curve was prepared to quantify the area under the curve for each sample

% Retinol at 8 Weeks

| | 25° C. | 45° C. |
|---|---|---|
| Composition A | 77% | 89% |
| Composition B | 81% | 78% |

The foregoing description illustrates and describes the invention. The disclosure shows and describes only the preferred embodiments but it should be understood that the invention is capable to use in various other combinations, modifications, and environments and is capable of changes or modifications within the scope of the inventive concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein.

### DEFINITIONS

As used herein, the term “fatty compound” includes silicone oils.

As used herein, the term “silicone oil” refers to a liquid polymerized siloxane compound having an organic side chain. There are various silicone oils depending on a type of the organic side chain.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the claimed invention.

The terms “a,” “an,” and “the” are understood to encompass the plural as well as the singular.

Thus, the term “a mixture thereof” also relates to “mixtures thereof.” Throughout the disclosure, if the term “a mixture thereof” is used, following a list of elements as shown in the following example where letters A-F represent the elements: “one or more elements selected from the group consisting of A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements chosen from A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

An “alkyl radical” is a linear or branched saturated hydrocarbon-based group, particularly C₁-C₈, more particularly C₁-C₆, preferably C₁-C₄ such as methyl, ethyl, isopropyl and tert-butyl;

An “alkoxy radical” is a alkyl-oxy wherein alkyl is as described herein before ;

An “alkenyl radical” is a linear or branched unsaturated hydrocarbon-based group, particularly C₂-C₈, more particularly C₂-C₆, preferably C₂-C₄ such as ethylenyl, propylenyl;

An “alkylene radical” is a linear or branched divalent saturated C₁-C₈, in particular C₁-C₆, preferably C₁-C₄ hydrocarbon-based group such as methylene, ethylene or propylene.

Some of the various categories of components identified for the cosmetic compositions may overlap. In such cases where overlap may exist and the composition/product includes two overlapping components (or more than two overlapping components), an overlapping component does not represent more than one component. As an example, a certain compound may be considered both a “fatty compound” and a “emulsifier.” If a particular composition/product includes both a fatty compound and an emulsifier, the compound can serve as only a fatty compound or as only the emulsifier (the single compound does not serve as both the compound and the emulsifier).

All percentages, parts and ratios herein are based upon the total weight of the compositions of the present invention, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub-ranges between, the given ranges. Thus, a range from 1-5, includes specifically points 1, 2, 3, 4 and 5, as well as sub-ranges such as 2-5, 3-5, 2-3, 2-4, 1-4, etc.; and points of 1, 2, 3, 4, and 5 includes ranges and sub-ranges of 1-5, 2-5, 3-5, 2-3, 2-4, 1-4, etc.

Other than in the operating examples, or where otherwise indicated, all numbers expressing quantities of ingredients and/or reaction conditions may be modified with the term “about,” whether or not expressly stated.

Additionally, all numbers are intended to represent exact values as additional embodiments, whether or not modified by the term “about.” For example, “an amount of about 1%” can be modified to refer to exactly 1%. As a further example, “an amount of 1%” can be modified to refer to “about 1%.” Unless otherwise indicated, the term “about” is understood to encompass a range of +/- 10% from the stated number. However, in some embodiments, the term may be defined to encompass narrower ranges, for example, +/- 1%, 2%, 3%, 4%, 5%, 6%, 7%, 8%, 9%, and 10% from the stated number.

The term “surfactants” and “emulsifiers” include salts of the surfactants and emulsifiers even if not explicitly stated. In other words, whenever the disclosure refers to a surfactant or emulsifier, it is intended that salts are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants and emulsifiers. However, additional cations such as ammonium ions, or alkanolammonium ions such as monoethanolammonium or triethanolammonium ions, may also form salts of surfactants.

The term “substantially free” or “essentially free” as used herein means the specific material may be present in small amounts that do not materially affect the basic and novel characteristics of the claimed invention. For instance, there may be less than 2% by weight of a specific material added to a composition, based on the total weight of the composition (provided that an amount of less than 2% by weight does not materially affect the basic and novel characteristics of the claimed invention). Similarly, a composition “substantially free” or “essentially free” of a stated material may include less than 1.5 wt.%, less than 1 wt.%, less than 0.5 wt.%, less than 0.1 wt.%, less than 0.05 wt.%, or less than 0.01 wt.%, or none of the specified material. The term “substantially free” or “essentially free” as used herein may also mean that the specific material is not added to the composition but may still be present in a raw material that is included in the composition.

Furthermore, all components that are positively set forth in the instant disclosure may be negatively excluded from the claims, e.g., a claimed composition may be “free,” “essentially free” (or “substantially free”) of one or more components that are positively set forth in the instant disclosure. As an example, the cosmetic composition of the instant disclosure may optionally include one or more phenolic or polyphenolic active compounds, for example, baicalin. However, in an embodiment, the cosmetic compositions may be free or essentially free from phenolic or polyphenolic active compounds, in particular baicalin.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cosmetic composition comprising:
(a) about 0.05 to about 6 wt.% of retinol;
(b) about 1 to about 8 wt.% of a plurality of nonionic emulsifiers comprising:
(i) one or more ethoxylated fatty acids;
(ii) one or more nonionic emulsifiers having a Hydrophile-Lipophile Balance (HLB) from about 9 to about 12;
(iii) optionally, one or more glyceryl esters; and
(iv) optionally, one or more additional nonionic emulsifiers;
(c) about 0.1 to about 8 wt.% of one or more fatty alcohols;
(d) about 1 to about 20 wt.% of one or more fatty compounds;
(e) one or more thickening polymers; and
(f) water;
wherein the composition is in the form of an oil in water emulsion and all percentages by weight are based on the total weight of the cosmetic composition.

2. The cosmetic composition of claim 1 comprising about 0.1 to about 3 wt.% of retinol.

3. The cosmetic composition of claim 1 comprising (b)(iii) one or more additional nonionic emulsifiers.

4. The cosmetic composition of claim 1, wherein the plurality of nonionic emulsifiers (b) comprises:
(i) about 0.1 to about 5 wt.% of the one or more glyceryl esters;
(ii) about 0.1 to about 5 wt.% of the one or more ethoxylated fatty acids; and
(iii) about 0.1 to about 5 wt.% of the one or more nonionic emulsifiers having a Hydrophile-Lipophile Balance (HLB) from about 9 to about 12; and
(iv) optionally, one or more additional nonionic emulsifiers.

5. The cosmetic composition of claim 1, wherein the glyceryl esters (b)(iii) are chosen from, glyceryl behenate, glyceryl erucate, glyceryl hydroxystearate, glyceryl isostearate, glyceryl lanolate, glyceryl laurate, glyceryl linoleate, glyceryl myristate, glyceryl palmitate lactate, glyceryl stearate, glyceryl distearate, glyceryl laurate, or a mixture thereof. In at least one instance the glyceryl ester comprises glyceryl stearate, glyceryl ricinoleate, and mixtures thereof.

6. The cosmetic composition of claim 1, wherein the ethoxylated fatty acids are chosen from adducts of ethylene oxide with esters of lauric acid, palmitic acid, stearic acid, or behenic acid, and mixtures thereof, comprising from 9 to 100 oxyethylene groups.

7. The cosmetic composition of claim 1, wherein the one or more nonionic emulsifiers having an HLB from about 9 to about 12 are chosen from alkylpolyglucosides (e.g., C12-20 alkyl glucoside), polyglycerol-based emulsifiers (e.g., polygyceryl-3 methylglucose distearate), sorbitan fatty esters (e.g., polysorbate 60), sugar fatty esters, polyol fatty esters, ethoxylates thereof, or a mixture thereof.

8. The cosmetic composition of claim 1, wherein the one or more fatty alcohols are chosen from fatty alcohols having from 8 to 24 carbon atoms, preferably chosen from C14-22 alcohols, cetyl alcohol, stearyl alcohol, cetearyl alcohol, behenyl alcohol, lauryl alcohol, myristic or myristyl alcohol, arachidyl alcohol, lignoceryl alcohol, and mixtures thereof.

9. The cosmetic composition of claim 1, wherein the one or more fatty compounds are chosen from fatty esters (e.g., diisopropyl sebacate, isononyl isononanoate), polyolefins (petrolatum), waxes, squalane, squalene, hydrogenated polyisobutene, hydrogenated polydecene, polybutene, mineral oil, pentahydrosqualene, plant and/or vegetable oil (e.g., soybean oil), hydrocarbon-based oils (isohexadecane), silicone oils (e.g., dimethicone, dimethiconol, and a mixture thereof.

10. The cosmetic composition of claim 1, wherein at least one of the one or more fatty compounds is a silicone oil, preferably dimethicone.

11. The cosmetic composition of claim 1, wherein the one or more water-soluble solvents are chosen from glycerin, mono-alcohols, polyols (polyhydric alcohols), glycols, and a mixture thereof.

12. The cosmetic composition of claim 1, wherein at least one of the one or more thickening polymers is a taurate copolymer.

13. The composition of claim 12, wherein the one or more taurate copolymers are chosen from acrylamide/sodium acryloyl dimethyl taurate copolymer, hydroxyethyl acrylate/sodium acryloyl dimethyl taurate copolymer, ammonium acryloyldimethyl taurate/VP copolymer, sodium acrylate/sodium acryloyl dimethyl taurate copolymer, hydroxyethyl acrylate/ sodium acryloyldimethyl taurate copolymer, and a mixture thereof.

14. The cosmetic composition of claim 1 comprising about 55 to about 85 wt.% of water.

15. The cosmetic composition of claim 1, further comprising:
(e) about 5 to about 20 wt.% of one or more water-soluble solvents.

16. The cosmetic composition of claim 1, further comprising:
(h) about 0.1 to about 10 wt.% of one or more miscellaneous ingredients.

17. The composition of claim 1 having a viscosity of about 20,000 to about 80,000 cPa.s at 25° C., and shear rate of 1 s⁻¹ at 25° C.

18. A cosmetic composition comprising or consisting of:
(a) about 0.4 to about 5 wt.% of retinol;
(b) about 1 to about 8 wt.% of a plurality of nonionic emulsifiers comprising:
(i) about 0.1 to about 5 wt.% of the one or more ethoxylated fatty acids;
(ii) about 0.1 to about 5 wt.% of the one or more nonionic emulsifiers having a Hydrophile-Lipophile Balance (HLB) from about 9 to about 12;
(i) optionally, about 0.1 to about 5 wt.% of the one or more glyceryl esters; and
(iv) optionally, one or more additional nonionic emulsifiers;
(c) about 0.5 to about 5 wt.% of one or more fatty alcohols chosen from fatty alcohols having from 8 to 24 carbon atoms;
(d) about 5 to about 10 wt.% of one or more fatty compounds;
(e) one or more thickening polymers chosen from taurate copolymers;
(f) about 55 to about 85 wt.% of water;
(g) about 5 to about 20 wt.% of one or more water-soluble solvents; and
(h) about 0.1 to about 8 wt.% of one or more miscellaneous ingredients chosen from miscellaneous emulsifiers/surfactants other than the nonionic emulsifiers of plurality of nonionic emulsifiers of (b), preservatives, fragrances, pH adjusters, salts, antioxidants, vitamins, botanical extracts, UV filtering agents, proteins, protein hydrolysates and/or isolates, hydrotropes, pearlescent agents, fillers, colorants, mattifying agents, further skin active agents, buffers, and mixtures thereof;
wherein the composition is in the form of an oil in water emulsion and all percentages by weight are based on the total weight of the cosmetic composition.

19. A kit comprising the cosmetic composition of claim 1 and one or more additional skin treatment compositions, wherein the cosmetic composition and each of the one or more skin treatment compositions are separately contained.

20. A method for treating skin comprising applying the cosmetic composition of claim 1 to the skin.
